(19) United States
(12) Patent Application Publication
(10) Pub. No.: US 2025/0164508 A1
(43) Pub. Date: May 22, 2025

(54) G-PROTEIN COUPLED RECEPTOR ASSAY

(71) Applicant: The Regents of the University of Michigan, Ann Arbor, MI (US)

(72) Inventors: Wenjing Wang, Ann Arbor, MI (US); Ruby Miller, Ann Arbor, MI (US)

(21) Appl. No.: 18/838,360

(22) PCT Filed: Feb. 16, 2023
(86) PCT No.: PCT/US2023/062766

§ 371 Date: Aug. 14, 2024

Related U.S. Application Data
(60) Provisional application No. 63/311,216, filed on Feb. 17, 2022.

(51) Int. Cl. G01N 33/74; C12Q 1/66; G01N 33/94

(52) U.S. Cl. CPC G01N 33/74; C12Q 1/66; G01N 33/9413; G01N 33/9486; G01N 2333/726; G01N 2500/04

(57) ABSTRACT

Provided herein are methods, compositions, kits and systems for in-solution assays of G-Protein Coupled Receptor (GPCR) activity. In particular, provided herein are methods, compositions, kits and systems comprising a fusion protein comprising a GPCR and a first nanoluciferase subunit, a GPCR conformation specific binder bound to a second nanoluciferase subunit wherein the GPCR conformation specific binder binds to an active GPCR, and a bioluminescent substrate to detect GPCR activation when the GPCR is bound to a ligand or drug in solution.

### CROSS-REFERENCE TO RELATED APPLICATIONS

The present application is a § 371 National Entry application of PCT/US2023/062766, filed Feb. 16, 2023, which claims priority to U.S. Provisional Application Ser. No. 63/311,216 filed Feb. 17, 2022, each of which are herein incorporated by reference in its entirety.

### STATEMENT REGARDING FEDERALLY SPONSORED RESEARCH OR DEVELOPMENT

This invention was made with government support under MH132939, and DA056192 awarded by the National Institutes of Health. The government has certain rights in the invention.

### SEQUENCE LISTING

The text of the computer readable sequence listing filed herewith, titled “39893_601_SequenceListing”, created Feb. 16, 2023, having a file size of 44,350 bytes, is hereby incorporated by reference in its entirety.

### FIELD

Provided herein are methods, compositions, kits and systems for in-solution assays of G-Protein Coupled Receptor (GPCR) activity. In particular, provided herein are methods, compositions, kits and systems comprising a fusion protein comprising a GPCR and a first nanoluciferase subunit, a GPCR conformation specific binder bound to a second nanoluciferase subunit wherein the GPCR conformation specific binder binds to an active GPCR, and a bioluminescent substrate to detect GPCR activation when the GPCR is bound to a ligand or drug in solution.

### BACKGROUND

G-protein coupled receptors (GPCRs) are critical participants in diverse cellular signaling pathways. (Irannejad, R.; Tomshine, J. C.; Tomshine, J. R.; Chevalier, M.; Mahoney, J. P.; Steyaert, J.; Rasmussen, S. G. F.; Sunahara, R. K.; El-Samad, H.; Huang, B.; von Zastrow, M. Conformational Biosensors Reveal GPCR Signalling from Endosomes. Nature 2013, 495 (7442), 534-538.) GPCRs function by converting an extracellular signal into intracellular signaling cascades by a ligand-induced transmembrane conformational change that leads to G-protein binding and activation. G-protein coupled receptors (GPCRs) are a class of seven transmembrane proteins that function as essential intracellular signal transducers. (Pierce, K. L., Premont, R. T. & Lefkowitz, R. J. Seven-transmembrane receptors. Nat Rev Mol Cell Biol 3, 639-650 (2002). Approximately 30% of FDA approved therapeutics target GPCRs. Santos, R. et al. A comprehensive map of molecular drug targets. Nat Rev Drug Discov 16, 19-34 (2017). Congreve, M., de Graaf, C., Swain, N. A. & Tate, C. G. Impact of GPCR Structures on Drug Discovery. Cell 181, 81-91 (2020). Due to the complexity of their signaling pathways and high modularity, GPCRs remain crucial targets for new therapeutic development. Wacker, D., Stevens, R. C. & Roth, B. L. How Ligands Illuminate GPCR Molecular Pharmacology. Cell 170, 414-427 (2017). Live cell-based assays have been instrumental for GPCR drug screening, as well as GPCR signaling and mechanistic studies. (Yasi, E. A., Kruyer, N. S. & Peralta-Yahya, P. Advances in G protein-coupled receptor high-throughput screening. Current Opinion in Biotechnology 64, 210-217 (2020), Siehler, S. Cell-based assays in GPCR drug discovery. Biotechnol. J. 3, 471-483 (2008). Martins, S. A. M. et al. Towards the miniaturization of GPCR-based live-cell screening assays, Trends in Biotechnology 30, 566-574 (2012), Haider, R. S., Godbole, A. & Hoffiann, C. To sense or not to sense new insights from GPCR-based and arrestin-based biosensors. Current Opinion in Cell Biology 57, 16-24 (2019), Sakamoto, S., Kiyonaka, S. & Hamachi, I. Construction of ligand assay systems by protein-based semisynthetic biosensors. Current Opinion in Chemical Biology 50, 10-18 (2019). Olsen, R. H. J. et al. TRUPATH, an open-source biosensor platform for interrogating the GPCR transducerome. Nat Chem Biol 16, 841-849 (2020). Olsen, R. H. J. & English, J. G. Advancements in G protein-coupled receptor biosensors to study GPCR-G protein coupling. British Journal of Pharmacology (2022), Horing, C. et al. A Dynamic, Split-Luciferase-Based Mini-G Protein Sensor to Functionally Characterize Ligands at All Four Histamine Receptor Subtypes. IJMS21, 8440 (2020), Hauge Pedersen, M. et al. A novel luminescence-based β-arrestin recruitment assay for unmodified receptors. Journal of Biological Chemistry 296, 100503 (2021), Inoue, A. et al. Illuminating G-Protein-Coupling Selectivity of GPCRs. Cell 177, 1933-1947.e1925 (2019).) However, there is still a lack of accessible and generalizable methods for detecting GPCR activation for broad applications. For example, it is infeasible with live cell assays to validate extracted GPCR protein's functionality for biochemical and structural studies.

As noted, over 34% of Food and Drug Administration (FDA) approved drugs target more than 100 members of the GPCR family. (Hauser A. S.; Chavali S.; Masuho I.; Jahn L. J.; Martemyanov K. A.; Gloriam D. E.; Babu M. M. Pharmacogenomics of GPCR Drug Targets. Cell. 2018 172, (1-2): 41-54.e19.) Over 830 different human genes comprising over 4% of the protein-coding genome are predicted to code for GPCRs based on genome sequence analysis. (Bjarnadóttir T. K.; Gloriam D. E.; Hellstrand S. H.; Kristiansson H.; Fredriksson R.; Schiöth H. B. Comprehensive repertoire and phylogenetic analysis of the G-protein-coupled receptors in human and mouse Genomics. 2006 88 (3): 263-73.) To identify therapeutic GPCRs, versatile and robust methods are needed to acquire information about how ligands activate GPCRs, and to screen ligands for agonist and antagonist discovery. (Laschet, C.; Dupuis, N.; Hanson, J. A Dynamic and Screening-Compatible Nanoluciferase-Based Complementation Assay Enables Profiling of Individual GPCR-G Protein Interactions. Journal of Biological Chemistry 2019, 294 (11), 4079-4090. Zeng, W.; Guo, L.; Xu, S.; Chen, J.; Zhou, J. High-Throughput Screening Technology in Industrial Biotechnology. Trends Biotechnol. 2020, 38 (8), 888-906.) A preferred screening platform is adaptable to test hundreds of thousands of candidate GPCR ligands. (Soave, M.; Heukers, R.; Kellam, B.; Woolard, J.; Smit, M. J.; Briddon, S. J.; Hill, S. J. Monitoring Allosteric Interactions with CXCR4 Using NanoBiT Conjugated Nanobodies. Cell Chem. Biol. 2020, 27(10), 1250-1261.)

Methods to test GPCR activation that exploit shared properties of GPCRs including GPCR structure, downstream signaling, and binding partners are labor-intensive and require use of toxic radioactive chemicals for drug screening. (Zhang, R.; Xie, X. Tools for GPCR Drug Discovery. Acta Pharmacol Sin 2012, 33 (3), 372-384. Manglik, A.; Kobilka, B. K.; Steyaert, J. Nanobodies to Study G Protein-Coupled Receptor Structure and Function. Annu. Rev. Pharmacol. Toxicol. 2017, 57 (1), 19-37.) Other approaches comprising radio-labeled ligand binding and activation-dependent fluorescent sensors require individualized engineering of each GPCR of interest. (Dixon, A. S.; Schwinn, M. K.; Hall, M. P.; Zimmerman, K.; Otto, P.; Lubben, T. H.; Butler, B. L.; Binkowski, B. F.; Machleidt, T.; Kirkland, T. A.; Wood, M. G.; Eggers, C. T.; Encell, L. P.; Wood, K. V. NANOLUC Complementation Reporter Optimized for Accurate Measurement of Protein Interactions in Cells. ACS Chem. Biol. 2016, 11 (2), 400-408.) Additional methods rely on detection of downstream signaling effects including production of cyclic-AMP (cAMP), as well as an increase in concentration of calcium (Ca²⁺). (Laschet, ibid) However, detection of downstream signaling events may result in non-specific and unreliable indicators of GPCR activation. Alternative assays employ ligand-induced protein interactions between a GPCR and a G-protein, or between a GPCR and arrestin. (Nuber, S.; Zabel, U.; Lorenz, K.; Nuber, A.; Milligan, G.; Tobin, A. B.; Lohse, M. J.; Hoffmann, C. β-Arrestin Biosensors Reveal a Rapid, Receptor-Dependent Activation/Deactivation Cycle. Nature 2016, 531 (7596), 661-664.) Though often used for in vivo screening of GPCR ligands, obstacles arise from adaptation of these method to high-throughput, live-cell screening platforms that require culture and handling of large batches of adherent cells while maintaining consistency between different batches of cells. A first obstacle is that culturing large batches of adherent cells for high-throughput screening (HTS) is time-consuming. A second obstacle is that in vivo GPCR assay results often vary due to changes in protein expression levels, and a lack of uniform membrane trafficking. Such a lack of consistency from batch to batch of cells results in inconsistent activation readouts. Accordingly, there is a need for in-solution assays for GPCR ligand HTS of greater efficiency, convenience in cell harvest and storage, uniformity of reagents, and consistency between screening assays.

### SUMMARY

Provided herein are methods, compositions, kits and systems for in-solution assays of G-Protein Coupled Receptor (GPCR) activity. In particular, provided herein are methods, compositions, kits and systems comprising a fusion protein comprising a GPCR and a first nanoluciferase subunit, a GPCR conformation specific binder bound to a second nanoluciferase subunit wherein the GPCR conformation specific binder binds to an active GPCR, and a bioluminescent substrate to detect GPCR activation when the GPCR is bound to a ligand or drug in solution.

In some embodiments, the present invention provides a method of measuring ligand activation of a G-protein coupled receptor (GPCR) in solution, comprising: expressing a GPCR fusion protein comprising a GPCR and a NANOLUC LgBit subunit or a NANOLUC SmBit subunit in a cell, extracting the GPCR fusion protein from the cell, generating a reaction mixture in solution comprising the GPCR fusion protein with a conformation specific nanobody wherein the nanobody is bound to a NANOLUC LgBit subunit, or NANOLUC SmBit subunit complementary to the NANOLUC SmBit subunit, or NANOLUC LgBit subunit fused to the GPCR, wherein binding of the NANOLUC LgBit to the NANOLUC SmBit generates a LgBit:SmBit luciferase, a bioluminescent substrate, and a buffer, adding the ligand to the reaction mixture in the solution, and measuring luminescence of the reaction mixture in the solution comprising the ligand wherein luminescence intensity indicates ligand activation of the GPCR. In some embodiments, the GPCR comprises a B2AR GPCR, a mu-opioid receptor (MOR) GPCR, or a dopamine receptor D1 (DRD1) GPCR. In some embodiments, the cell is a prokaryotic cell or a mammalian cell. In some embodiments, the conformation specific nanobody comprises Nb80, Nb39 or Nb40. In some embodiments, the NANOLUC LgBit subunit is 17.5 kDa. In some embodiments, the NANOLUC SmBit comprises 11 amino acids.

In some embodiments, the present invention provides method of measuring ligand activation of a G-protein coupled receptor (GPCR) in solution, comprising: expressing a GPCR fusion protein comprising the GPCR and a NANOLUC LgBit subunit or a NANOLUC SmBit subunit in a cell, extracting the GPCR fusion protein from said cell, generating a reaction mixture in solution comprising the GPCR fusion protein with a conformation specific peptidomimetic wherein the conformation specific peptidomimetic is bound to a NANOLUC LgBit subunit, or NANOLUC SmBit subunit complementary to the NANOLUC SmBit subunit, or NANOLUC LgBit subunit fused to the GPCR, wherein binding of the NANOLUC LgBit to the NANOLUC SmBit generates a LgBit:SmBit luciferase, a bioluminescent substrate, and a buffer, adding the ligand to the reaction mixture in the solution; and measuring luminescence of the reaction mixture in the solution comprising the ligand wherein luminescence intensity indicates ligand activation of the GPCR. In some embodiments, the GPCR comprises a beta-2-adrenergic receptor (B2AR) GPCR, a mu-opioid receptor (MOR) GPCR, or a dopamine receptor D1 (DRD1) GPCR. In some embodiments, the cell is a prokaryotic cell or a mammalian cell. In some embodiments, the conformation specific peptidomimetic is selected from the group consisting of SEQ ID NO.: 1 to SEQ ID NO.: 46. In some embodiments, the NANOLUC LgBit subunit is 17.5 kDa. In some embodiments, the NANOLUC SmBit comprises 11 amino acids.

In some embodiments, the present invention provides a kit, comprising: a GPCR fusion protein comprising the GPCR and a NANOLUC LgBit subunit or a NANOLUC SmBit: a conformation specific nanobody wherein the nanobody is bound to a NANOLUC LgBit subunit or NANOLUC SmBit subunit complementary to the NANOLUC LgBit subunit or NANOLUC SmBit subunit fused to the GPCR. a bioluminescent substrate; and a buffer. In some embodiments, the kit comprises one or more test ligands and/or one or more control ligands.

In some embodiments, the present invention provides a kit, comprising: a GPCR fusion protein comprising the GPCR and a NANOLUC LgBit subunit or a NANOLUC SmBit: a conformation specific peptidomimetic wherein the conformation specific peptidomimetic is bound to a NANOLUC LgBit subunit or NANOLUC SmBit subunit complementary to the NANOLUC LgBit subunit or NANOLUC SmBit subunit fused to the GPCR. a bioluminescent substrate; and a buffer. In some embodiments, the kit comprises one or more test ligands and/or one or more control ligands.

In some embodiments, the present invention provides a composition, comprising a GPCR fusion protein comprising the GPCR and a NANOLUC LgBit subunit or a NANOLUC SmBit, a conformation specific nanobody, wherein the nanobody is bound to a NANOLUC LgBit subunit or NANOLUC SmBit subunit complementary to the NANOLUC LgBit subunit or NANOLUC SmBit subunit fused to the GPCR, a bioluminescent substrate, and a buffer. In some embodiments the composition comprises one or more test ligands and/or one or more control ligands.

In some embodiments, the present invention provides a composition, comprising a GPCR fusion protein comprising the GPCR and a NANOLUC LgBit subunit or a NANOLUC SmBit, a conformation specific peptidomimetic, wherein the conformation specific peptidomimetic is bound to a NANOLUC LgBit subunit or NANOLUC SmBit subunit complementary to the NANOLUC LgBit subunit or NANOLUC SmBit subunit fused to the GPCR, a bioluminescent substrate, and a buffer. In some embodiments the composition comprises one or more test ligands and/or one or more control ligands.

In some embodiments, activated-GPCR-conformation-specific binders and G-protein mimics, including nanobodies, antibodies, mini-Gs proteins and peptidomimetics are bound to the split NANOLUC subunits to assay GPCR activity. In some embodiments, any protein that selectively binds with greater affinity to the activated form of the GPCR find use in the assay. In some embodiments, nanobodies are bound to either the N- or C-terminus of the split NANOLUC subunits. In some embodiments, mini-G proteins and G-protein peptidomimetics are fused to the C-terminus of the split NANOLUC subunits such that their C-terminus is free of fusion to interact with an activated GPCR.

In some embodiments, the present invention provides in vitro assay with a detergent-free solubilization buffer to resuspend the cell-pellet, and sonication to lyse the cells to prepare a homogeneous cell lysate containing the native GPCR membrane component. In some embodiments, when using a peptidomimetic as a conformation-specific binder, a GPCR membrane component and a SmBit-peptidomimetics fusion peptide solution are prepared separately and mixed during assays. In some embodiments, when using nanobodies and mini-G proteins as conformation specific binders, the nanobody-SmBit and SmBit-mini-G protein fusion proteins are co-expressed together as a single reagent or expressed separately as two reagents.

In some embodiments, the assay is performed with a saturated concentration of agonists in parallel with a no-drug control group comprising vehicle only. In some embodiments, a T-test is used to compare the 2 groups of samples. If a >1-fold drug-dependent luminescence increase and statistical significance (p<0.05) using student T-test is achieved, the reagents and conditions meet performance standards.

In some embodiments, the present invention provides a generalizable and accessible In vitro GPCR split NANOLUC ligand Triggered Reporter (IGNiTR), having broad and diverse applications. IGNiTR leverages the interaction between a conformation-specific binder and agonist-activated GPCR to reconstitute a split nanoluciferase. In some embodiments, IGNiTR comprises three Gₛ-coupled GPCRs and a Gᵢ-coupled GPCR with three classes of conformation-specific binders: nanobodies, miniG proteins, and G-protein peptidomimetics. IGNiTR demonstrates binding efficacy and potency values of diverse Dopamine Receptor D1 (DRD1) ligands. IGNiTR further supports use of a synthetic G protein peptidomimetic, thereby providing easily standardized reagents for characterizing GPCRs and ligands. In some embodiments IGNiTR finds use in: 1) characterizing GPCR functionality during Nanodisc-based reconstitution process; 2) high-throughput screening of ligands against DRD1; and 3) detection of opioids for in the field applications. Because of convenience, accessibility and consistency, IGNiTR supports extensive applications in GPCR ligand detection, screening and GPCR characterization.

In some embodiments, the present invention provides an in vitro assay, based on GPCR protein in cell lysate in a simple and easily adaptable format for broad applications. Existing in vitro assays, including radioligand binding, monitor GPCR-ligand binding, but do not measure ligand efficacy for inducing the active conformation, which recruits downstream G-proteins. (Yasi, E. A., Kruyer, N. S. & Peralta-Yahya, P. Advances in G protein-coupled receptor high-throughput screening. Current Opinion in Biotechnology 64, 210-217 (2020), Wiseman, D. N. et al. Expression and purification of recombinant G protein-coupled receptors: A review. Protein Expression and Purification 167, 105524 (2020), Enrico Rovati, G. Ligand-binding studies: old beliefs and new strategies. Trends in Pharmacological Sciences 19, 365-369 (1998). Masureel, M. et al. Structural insights into binding specificity, efficacy and bias of a β2AR partial agonist. Nat Chem Biol 14, 1059-1066 (2018). Keen, M. in Signal Transduction Protocols Vol. 41 1-16 (Humana Press, 1995), Saumell-Esnaola, M. et al. Design and validation of recombinant protein standards for quantitative Western blot analysis of cannabinoid CB1 receptor density in cell membranes: an alternative to radioligand binding methods. Microb Cell Fact 21, 192 (2022), Volz, M. R. & Moosmann, B. Development of a non-radioactive mass spectrometry-based binding assay at the μ-opioid receptor and its application for the determination of the binding affinities of 17 opiates/opioids as well as of the designer opioid isotonitazene and five further 2-benzylbenzimidazoles. Analytica Chimica Acta 1219, 339978 (2022).) An alternative approach, reconstitution of split bioluminescent enzymes, is used to report on protein-protein interactions. We harness the robust luminescent signal that is quantifiable in a complex biological environment to track a ligand-induced binding interaction. (Soave, M. et al. Monitoring Allosteric Interactions with CXCR4 Using NanoBiT Conjugated Nanobodies. Cell Chemical Biology 27, 1250-1261.e1255 (2020), Israeli, H. et al. Structure reveals the activation mechanism of the MC4 receptor to initiate satiation signaling. Science 372, 808-814 (2021). Laschet, C., Dupuis, N. & Hanson, J. A dynamic and screening-compatible nanoluciferase-based complementation assay supports profiling of individual GPCR-G protein interactions. Journal of Biological Chemistry 294, 4079-4090 (2019).)

In some embodiments, IGNiTR leverages agonist-dependent GPCR conformational change and subsequent recruitment of G-proteins and other conformation-specific binders (Roth, B. L., Irwin, J. J. & Shoichet, B. K. Discovery of new GPCR ligands to illuminate new biology. Nat Chem Biol 13, 1143-1151 (2017), Weis, W. I. & Kobilka, B. K. The Molecular Basis of G Protein-Coupled Receptor Activation. Annu. Rev. Biochem. 87, 897-919 (2018).) to reconstitute split nanoluciferase (NANOLUC) (Roth, B. L., Irwin, J. J. & Shoichet, B. K. Discovery of new GPCR ligands to illuminate new biology. Nat Chem Biol 13, 1143-1151 (2017), Weis, W. I. & Kobilka, B. K. The Molecular Basis of G Protein-Coupled Receptor Activation. Annu. Rev. Biochem. 87, 897-919 (2018).) (Dixon, A. S. et al. NANOLUC Complementation Reporter Optimized for Accurate Measurement of Protein Interactions in Cells. ACS Chem. Biol. 11, 400-408 (2016). Ni, Y., Arts, R. & Merkx, M. Ratiometric Bioluminescent Sensor Proteins Based on Intramolecular Split Luciferase Complementation. ACS Sens. 4, 20-25 (2019).) Unlike live cell assays, IGNiTR components are easily stored with cell pellets expressing GPCR components frozen to preserve the integral, native lipid environment. Additionally, IGNiTR allows the use of peptidomimetics as conformation-specific binders, broadening assay applications.

In some embodiments, IGNiTR supports three applications. First, IGNiTR provides a robust platform for high-throughput screening of ligands against Dopamine Receptor D1. Second, IGNiTR rapidly detects the μ-opioid receptor agonist fentanyl at nanomolar range in an easy and portable setup for potential field applications. Third, IGNiTR is used characterize protein functionality of GPCR protein samples at different stages of the Nanodisc-based GPCR extraction and reconstitution process. IGNiTR's adaptability supports unique applications complementary to live cell assays or existing in vitro assays.

### DESCRIPTION OF THE FIGURES

FIG. 1 shows an overview of the in-solution, cell-free assay using a nanobody, mini-G protein or peptidomimetic conformation specific binder. A GPCR protein is expressed in fusion with a small-Bit peptide (SmBit) or large-bit nanoluciferase protein fragment (LgBit split) NANOLUC component and prepared from cells. A conformation specific binder is prepared in fusion with the complementary SmBit or LgBit component in cells (e.g., mammalian cells and bacterial cells), or is synthesized. The GPCR and conformation specific binder together with the complementary SmBit and LgBit components are mixed in the in-solution assay.

FIG. 2 shows a cell culture assay of the effects SmBit/LgBit binding on either the GPCR or the conformation specific binder has on the luminescent signal fold change. Shown are 2 versions with the fragments of split NANOLUC attached to both B2AR and Nb80. Isoproterenol=[9 uM].

FIG. 3 shows a cell culture assay of luminescent signal fold change for mu-opioid receptor (MOR)-SmBit binding with both Nb39 and Nb44 in the presence and absence of fentanyl [9 μM].

FIG. 4 shows an in-solution assay of signal fold change for B2AR and Nb80 in cell lysate. Isoproterenol=[8.75 μM]

FIG. 5 shows an in-solution assay of signal fold change for B2AR (cell lysate) and a Gₛ-mimic hybrid peptide VTGYRLFEEILGSKKKFN-C-SRD-C-IQRMHLRQYE-{Cha}-L (SEQ ID NO.: 1) [2.5 μM]. Isoproterenol=[8.75 μM]“Cha” indicates a non-natural cyclic residue.

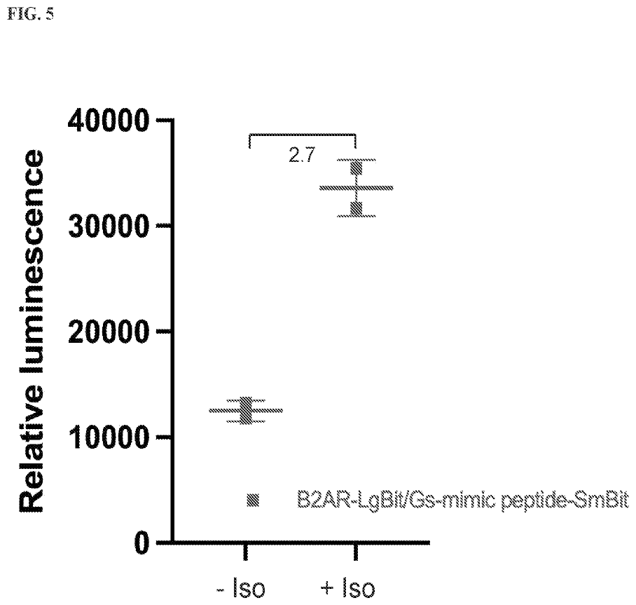

FIG. 6 shows a protocol for a GPCR activation-based luminescent assay of the present invention using a nanobody.

FIG. 7 shows a signal output for an activated B2AR GPCR binding to nanobody Nb80 assay with a significant difference in binding in the presence and absence of isoproterenol. (****=p<0.0001.)

FIG. 8 shows a signal output for an activated MOR GPCR binding to nanobody Nb39 assay with a significant difference in binding in the presence and absence of loperamide (****=p<0.0001.)

FIG. 9 shows a protocol for a GPCR activation-based luminescent assay of the present invention using a peptidomimetic.

FIG. 10 shows a signal output for an activated B2AR GPCR binding to a Gs peptidomimetic (VTGYRLFEEILGSFNDCRDIIQRMHLRQYE-{Cha}-L) (SEQ ID NO.: 2) assay with a significant difference in binding in the presence and absence of isoproterenol. (***=p<0.001.)

FIG. 11 shows a signal output for an activated dopamine receptor D1 (DRD1) GPCR binding to a Gs peptidomimetic (VTGYRLFEEILGSFNDCRDIIQRMHLRQYE-{Cha}-L) (SEQ ID NO.: 2) assay with a significant difference in binding in the presence and absence of dopamine. (***=p<0.0001.)

FIG. 12 shows G-protein C terminal helix peptidomimetic amino acid sequences comprising a leucine residue.

FIG. 13 shows a set of SmBit-G protein peptidomimetic fusion protein amino acid sequences comprising the G-protein C terminal helix peptidomimetic amino acid sequences of FIG. 13 comprising a leucine residue.

FIG. 14. shows G-protein C terminal helix peptidomimetic amino acid sequences comprising a non-natural Cha (cyclohexylamine) residue.

FIG. 15 shows a set of SmBit-G protein peptidomimetic fusion protein amino acid sequences comprising the G-protein C terminal helix peptidomimetic amino acid sequences of FIG. 14 comprising a Cha residue.

FIG. 16 shows successful binding of a Gᵢ-peptidomimetic to the MOR protein. Columns on the left represent a peptidomimetic (VTGYRLFEEILGSKKKFDAVTDIIIKNNLKDCG-{Cha}-F) (SEQ ID NO.: 3) concentration of [35 uM] and columns on the right represent [28 uM].

FIG. 17 shows a flow chart for the co-expression of GPCR-SmBit and Nb-LgBit protein components.

FIG. 18 shows MOR GPCR activation by loperaminde in separately expressed pellets containing MOR-SmBit and Nb39-LgBit (left), and MOR GPCR activation by loperaminde in co-expressed pellets with both MOR-SmBit and Nb39-LgBit.

FIG. 19 shows a characterization of IGNiTR with MiniGₛ and a conformation-specific nanobody. A. Schematics of the IGNiTR assay: LgBiT attached to a GPCR and SmBiT attached to a conformation-specific binder. Ligand activation of a GPCR results in GPCR-conformation specific binder interaction, triggering split NANOLUC reconstitution. B. Characterization of IGNiTR with β2AR. C. DRD1, and D. MC4R with Nanobody 80 (Nb80) and MiniGₛ as the conformation specific binder. Drug, 10 μM. RLU: Relative Luminescent Units. n=3 for β2AR and n=6 for MC4R and DRD1. Values above the bars represent the DDR. Stars indicate significance after performing an unpaired Student's t-test. ***P≤0.001, ****P≤0.0001. “n.s.” indicates no significant difference between the two conditions.

FIG. 20 shows a comparison of IGNiTR component fusion geometry prepared in no detergent vs detergent conditions. Four versions of IGNiTR with different fusion geometries and Nb80 or miniG as conformation-specific binder were tested. The cell pellet expressing these IGNiTR components were lysed by sonication in solutions with or without the detergent mix (1% DDM (n-dodecyl β-D-maltoside) and 0.1% CHS (cholesteryl hydrogen succinate). Addition of detergent significantly decreases luminescence in all conditions. Therefore, we used sonication without detergent in the remaining work. Numbers inside the grids are relative luminescence signal.

FIG. 21 shows A. Amino acid sequences of the fusion peptides. B. Model structure based on LY3154207-bound DRD1 (PDB: 7X2F). Mutation phenylalanine is introduced in the penultimate position of the Gₛ protein's α-5-helix to illustrate the interaction between the α-5-helix with the hydrophobic binding pocket of the activated DRD1. C. Comparison of DRD1-IGNiTR signal with a panel of drugs at saturated concentrations. SCH23390, 50 μM; SCH23390+Dopa (Dopamine), 50 and 10 μM; Dopa, 10 μM; Fen (Fenaldopam), 10 μM. Luminescence values were acquired 30 minutes post drug incubation.

FIG. 22 shows a flowchart showing preparation of IGNiTR assay. MRB is membrane resuspension buffer.

FIG. 23 shows a characterization of the IGNiTR assay with peptidomimetics. A. Characterization of the IGNiTR assay with the Gₛ fusion peptide for β2AR, MC4R and DRD1. Gₛ fusion peptide, 2 μM; drug, 10 μM. n=6. B. Characterization of μ-OR IGNiTR with the G; fusion peptide. n=3. Values above the bars represent the DDR. C. Dose-response curve of DRD1 IGNiTR with dopamine, fenoldopam and SCH 23390. For dopamine, EC50 range within 95% confidence is 1.8 to 4.1 μM. For fenoldopam, EC50 range within 95% confidence is 114 nM to 181 nM. For SCH 23390, IC50 range within 95% confidence is 20 nM to 33 nM. n=4. Stars indicate significance after performing an unpaired Student's t-test **** P<0.0001.

FIG. 24 shows a standard curve using the luminescent signal of the high-affinity binder HiBiT with known concentrations of MBP-LgBiT to determine the concentration of DRD1-LgBiT.

FIG. 25 shows the time course of the luminescent signal of DRD1-LgBiT with different concentrations of peptidomimetics. Time 0, when drug is added. 10 μM dopamine was used.

FIG. 26 shows characterization of the IGNiTR assay with Gₛ and Gᵢ fusion peptides. A. Characterization of the effects of GPCR cell lysate dilution factors on IGNiTR using DRD1. B. Characterization of the μ-OR-based IGNiTR with cell lysate in a range of dilutions. C. Characterization of the effects of a range of Gₛ peptidomimetic concentrations on IGNiTR using DRD1-LgBiT. DDR were taken around 30 minutes after drug incubation for both. N=6 D. Characterization of the effects of a range of Gᵢ peptidomimetic concentrations on IGNiTR using μ-OR-LgBiT. DDR were taken around 10 minutes after drug incubation for both. n=3.

FIG. 27 shows Z′ values measured across a proof-of-concept drug screening for DRD1-LgBiT and Gₛ fusion peptide across 6×384-well plates.

FIG. 28 shows applications of IGNiTR. A. Imaging and B. quantification of the IGNiTR assay performed with μ-OR LgBiT and the Gᵢ fusion peptide to detect varied concentrations of fentanyl. n=4. C. Workflow for the incorporation of β2AR-LgBiT into POPC-based Nanodiscs. “NTA” represents Ni-NTA column purification. D. Analysis of the β2AR-LgBiT samples in C using IGNiTR with Gₛ fusion peptide. Stars indicate significance after performing an unpaired Student's t-test. ****P value <0.0001. ***P value <0.001.

### DEFINITIONS

To facilitate an understanding of the present disclosure, a number of terms and phrases are defined below. While the invention will be described in conjunction with certain representative embodiments, it will be understood that the invention is not limited to these illustrative examples. One skilled in the art will recognize many methods and materials similar or equivalent to those described herein may be used in the practice of the present invention. Unless defined otherwise, technical and scientific terms used herein have the meaning commonly understood by one of ordinary skill in the art to which this invention belongs. Although any methods, devices, and materials similar or equivalent to those described herein can be used in the practice of the invention, certain methods, devices, and materials are described herein. It is further to be understood that all base sizes or amino acid sizes, and all molecular weight or molecular mass values, given for nucleic acids or polypeptides are approximate, and are provided for description.

As used in this disclosure, including the appended claims, the singular forms “a,” “an,” and “the” include plural references, unless the content clearly dictates otherwise, and are used interchangeably with “at least one” and “one or more.”

As used herein, the term “about” represents an insignificant modification or variation of the numerical value such that the basic function of the item to which the numerical value relates is unchanged.

As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

As used herein, “modulate” means to alter, either by increasing or decreasing, the activity of a gene or protein. The term “inhibit”, as used herein, means to prevent or reduce the activity of gene or protein.

As used herein, the term “bioactivity” indicates an effect on one or more cellular or extracellular process (e.g., via binding, signaling, etc.) that can impact physiological or pathophysiological processes.

Ranges provided herein are understood to be shorthand for all of the values within the range. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell lysate. The term “in vivo” refers to the natural environment (e.g., an animal or a cell) and to processes or reaction that occur within a natural environment.

### DETAILED DESCRIPTION OF THE DISCLOSURE

Provided herein are methods, compositions, kits and systems for in-solution assays of G-Protein Coupled Receptor (GPCR) activity. In particular, provided herein are methods, compositions, kits and systems comprising a fusion protein comprising a GPCR and a first nanoluciferase subunit, a GPCR conformation specific binder bound to a second nanoluciferase subunit wherein the GPCR conformation specific binder binds to an active GPCR, and a bioluminescent substrate to detect GPCR activation when the GPCR is bound to a ligand or drug in solution.

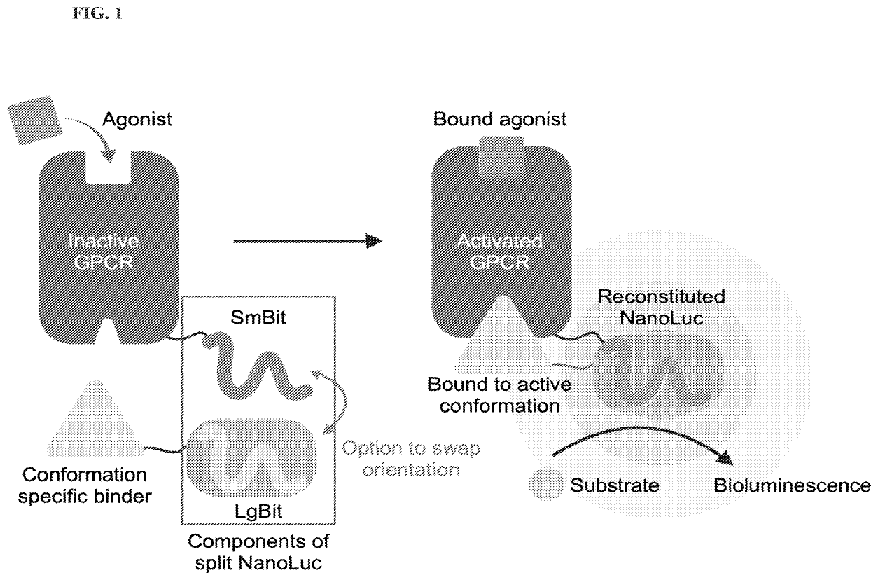

In some embodiments, the present invention provides versatile methods, compositions, systems and kits to perform an in-solution assay that serves as a robust platform to screen GPCR ligands, and to characterize activation of GPCR proteins isolated from the membrane. Some embodiments comprise a quantifiable, bioluminescent signal coupled to the conformational change of a GPCR induced by ligand binding (FIG. 1). Because the readout depends upon whether a GPCR undergoes the conformational changes with receptor activation, the assay is of use to verify GPCR functionality in-solution. Generalizability of the assay provides platform to screen a diversity of GPCRs and ligands.

In some embodiments, the present invention comprises conformation-specific binders that bind only to the active state of the GPCR to detect GPCR activity. Some embodiments comprise reconstitution of the protein interaction-dependent split protein using the bioluminescent enzyme nanoluciferase (NANOLUC, Promega, Madison, WI). (Dixon, A. S.; Schwinn, M. K.; Hall, M. P.; Zimmerman, K.; Otto, P.; Lubben, T. H.; Butler, B. L.; Binkowski, B. F.; Machleidt, T.; Kirkland, T. A.; Wood, M. G.; Eggers, C. T.; Encell, L. P.; Wood, K. V. NANOLUC Complementation Reporter Optimized for Accurate Measurement of Protein Interactions in Cells. ACS Chem. Biol. 2016, 11 (2), 400-408, Ni, Y.; Arts, R.; Merkx, M. Ratiometric Bioluminescent Sensor Proteins Based on Intramolecular Split Luciferase Complementation. ACS Sens. 2019, 4 (1), 20-25.) On activation of the GPCR by an agonist, a conformation-specific binder binds to the active-state GPCR and reconstitutes the split NANOLUC. (Soave, M.; Heukers, R.; Kellam, B.; Woolard, J.; Smit, M. J.; Briddon, S. J.; Hill, S. J. Monitoring Allosteric Interactions with CXCR4 Using NanoBiT Conjugated Nanobodies. Cell Chem. Biol. 2020, 27(10), 1250-1261.) In some embodiments, in the presence of the substrate furimazine the reconstituted enzyme produces a quantifiable bioluminescent signal. (Dixon, ibid, Ni, ibid.) Rapid reconstitution of split NANOLUC and the resulting bioluminescent signal produces a quantifiable indicator of GPCR functionality and agonist activation in solution. In some embodiments, the present invention provides an in-solution assay for the characterization and quantification of GPCR activation, and for screening candidate activating, inactivating, and neutral ligands. Advantages of an in-solution assay over existing technologies include: 1) increased efficiency of GPCR expression in suspended mammalian cells on a large scale compared to adherent cells; 2) expression of GPCRs in different batches of cells; and 3) extraction of the proteins and their combination antecedent to aliquoting for different assays to enhance assay consistency. Mixing of the proteins before aliquoting provides uniformity of reagents, and greater consistency between different experiments than can be achieved using live cell protocols. Because the GPCR protein extract may be harvested and stored in advance, reduced is required for cell culture. In some embodiments, the GPCRs are isolated from cell lysate. Accordingly, the split NANOLUC-based in-solution assays of the present invention provide a cost-effective and convenient platform to characterize the functionality, folding and activation of diverse membrane-extracted GPCRs.

In some embodiments, the present invention provides a generalizable in vitro GPCR assay, IGNiTR, able to characterize a GPCR's structural integrity and activity by detecting the agonist-induced interaction of the GPCR with a conformation-specific binder. IGNiTR comprises features that are both complementary to and advantageous over live cell-based assays. First, IGNiTR components, including the GPCR and the conformation-specific binder components may be prepared in advance and stored frozen until usage. Second, IGNiTR may be performed without the restrictions of working with live mammalian cells following biosafety level 2 regulations. Third, the preparation of IGNiTR in a cell lysate solution supports use of a synthetic fusion G protein peptidomimetic, with concentrations that are well-controlled for assay fine-tuning, including optimization of DDR. Fourth, mixing of the components supports standardization of the reaction conditions in thousands of wells to achieve consistency across HTS plates. IGNiTR provides multiple advantages over existing in vitro assays. IGNiTR's bioluminescent readout is quantifiable in a single step, and therefore is easily scaled up and performed in the field while existing in vitro GPCR assays, including the radioligand assay, require a complex protocol and platform. In some embodiments, the present invention support diverse applications in, or example: 1) HTS of GPCR ligands; 2) characterization and detection of GPCR ligands in the lab and in the field; and 3) verification of GPCR structural integrity antecedent to and during in vitro diverse GPCR characterizations.

### Nanobody Conformation-Specific Binders for In-Solution Ligand Screening.

In some embodiments, the present invention provides in-solution GPCR ligand screening using conformation-specific binders. In some embodiments, the conformation-specific binder comprises one or more nanobodies. In some embodiments, the GPCR is a Mu-opioid receptor (MOR). MORs regulate pain modulation. Opioid molecules that target MORs comprise the most potent pain medications. (Darcq, E.; Kieffer, B. L. Opioid Receptors: Drivers to Addiction? Nat. Rev. Neurosci. 2018, 19 (8), 499-514.) However, available synthetic opioids have severe side effects, including addiction and ventilatory depression. (Wang, Y.; Fan, Z.; Shao, L.; Kong, X.; Hou, X.; Tian, D.; Sun, Y.; Xiao, Y.; Yu, L. Nanobody-Derived Nanobiotechnology Tool Kits for Diverse Biomedical and Biotechnology Applications. Int. J. Nanomedicine 2016, Volume 11, 3287-3303.) Conventional in vivo assays for screening opioids rely on late-stage events in the MOR signaling cascade. A robust and easily scalable in-solution assay for screening opioid ligands by detecting the early-stage signaling events is needed to screen alternative non-addictive opioids. In some embodiments, the present invention provides in-solution MOR ligand screening assay. As shown in FIG. 1, the in-solution split NANOLUC assay comprises conformation-specific binders. In some embodiments, the present invention provides conformation-specific binder nanobodies. Nanobodies may be derived from single-domain antibodies found in llamas that are ˜120 amino acids in length and may be expressed and purified in E. coli. (Manglik, ibid.) Nanobodies may be designed to mimic G-protein binding to stabilize the active conformation of the highly dynamic GPCRs for crystallography. (Irannejad, R.; Tomshine, J. C.; Tomshine, J. R.; Chevalier, M.; Mahoney, J. P.; Steyaert, J.; Rasmussen, S. G. F.; Sunahara, R. K.; El-Samad, H.; Huang, B.; von Zastrow, M. Conformational Biosensors Reveal GPCR Signalling from Endosomes.) For example, the conformation specific nanobody, Nanobody 80 (Nb80), is designed for the crystallization of agonist bound B2AR. (Rasmussen, S. G. F.; DeVree, B. T.; Zou, Y.; Kruse, A. C.; Chung, K. Y.; Kobilka, T. S.; Thian, F. S.; Chae, P. S.; Pardon, E.; Calinski, D.; Mathiesen, J. M.; Shah, S. T. A.; Lyons, J. A.; Caffrey, M.; Gellman, S. H.; Steyaert, J.; Skiniotis, G.; Weis, W. I.; Sunahara, R. K.; Kobilka, B. K. Crystal Structure of the B2 Adrenergic Receptor-Gs Protein Complex. Nature 2011, 477(7366), 549-555, Rasmussen, S. G. F.; Choi, H.-J.; Fung, J. J.; Pardon, E.; Casarosa, P.; Chae, P. S.; DeVree, B. T.; Rosenbaum, D. M.; Thian, F. S.; Kobilka, T. S.; Schnapp, A.; Konetzki, I.; Sunahara, R. K.; Gellman, S. H.; Pautsch, A.; Steyaert, J.; Weis, W. I.; Kobilka, B. K. Structure of a Nanobody-Stabilized Active State of the B2 Adrenoceptor. Nature 2011, 469 (7329), 175-180, Ring, A. M.; Manglik, A.; Kruse, A. C.; Enos, M. D.; Weis, W. I.; Garcia, K. C.; Kobilka, B. K. Adrenaline-Activated Structure of B2-Adrenoceptor Stabilized by an Engineered Nanobody. Nature 2013, 502 (7472), 575-579.) Nb39 is designed to bind specifically to the opioid-activated MOR. (Huang, W.; Manglik, A.; Venkatakrishnan, A. J.; Laeremans, T.; Feinberg, E. N.; Sanborn, A. L.; Kato, H. E.; Livingston, K. E.; Thorsen, T. S.; Kling, R. C.; Granier, S.; Gmeiner, P.; Husbands, S. M.; Traynor, J. R.; Weis, W. I.; Steyaert, J.; Dror, R. O.; Kobilka, B. K. Structural Insights into M-Opioid Receptor Activation. Nature 2015, 524 (7565), 315-321, Vasudevan, L.; Stove, C. P. A Novel Nanobody-Based Bio-Assay Using Functional Complementation of a Split Nanoluciferase to Monitor Mu-Opioid Receptor Activation. Anal. Bioanal. Chem. 2020, 412 (29), 8015-8022. Stoeber, M.; Jullié, D.; Lobingier, B. T.; Laeremans, T.; Steyaert, J.; Schiller, P. W.; Manglik, A.; von Zastrow, M. A Genetically Encoded Biosensor Reveals Location Bias of Opioid Drug Action. Neuron 2018, 98 (5), 963-976.e5.) Conformation-specific nanobodies of the present invention, also termed “single variable domain antibodies”, are small in size, for example, about 15 kDa. In some embodiments, nanobodies of the present invention have a structure that comprises β-barrel constant regions and 3 variable loops that form the complementary determining regions (CDRs). (Muyldermans, S. Nanobodies: Natural Single-Domain Antibodies. Annu. Rev. Biochem. 82, 775-797 (2013).) The highly variable CDRs constitute nanobody binding sites.

### Peptidomimetic Conformation-Specific Binders for In-Solution Ligand Screening

In some embodiments, the present invention provides in-solution GPCR ligand screening methods, compositions, systems and kits comprising G-protein peptidomimetic conformation-specific binders. In some embodiments, a G-protein peptidomimetic is fused with LgBit of the NANOLUC Assay, and SmBit of the NANOLUC assay is fused with a GPCR. In some embodiments, a G-protein peptidomimetic is fused with SmBit of the NANOLUC Assay, and LgBit of the NANOLUC assay is fused with a GPCR. G-protein peptidomimetic conformation-specific binders support in-solution assays of the present invention that are adaptable with a broad diversity of GPCRs of value in high-throughput ligand screening. Use of G-protein peptidomimetics is highly advantageous given its capacity to use unnatural amino acids to achieve increased stability and higher binding affinity for the GPCR target. (Kapolka, N. J.; Taghon, G. J.; Rowe, J. B.; Morgan, W. M.; Enten, J. F.; Lambert, N. A.; Isom, D. G. DCyFIR: A High-Throughput CRISPR Platform for Multiplexed G Protein-Coupled Receptor Profiling and Ligand Discovery. Proc. Natl. Acad. Sci. 2020, 117(23), 13117.) In turn, peptidomimetic conformation-specific binders provide economic advantages that arise from the low molecular weight of the peptide, and use of peptide synthesis to bypass protein expression and purification. In some embodiments, a 10 mg peptidomimetic synthesis may be used for at least 10,000 assay reactions. Accordingly, peptidomimetic conformation-specific binders for in-solution ligand screening transform the GPCR screening process to identify candidate agonists for GPCR targets including, for example, GPCRs that participate in drug dependency.

### Kits

The present disclosure provides kits comprising components of the GPCR ligand binding assays described herein. Such kits may comprise, for example, two or more of 1) at least one conformation specific nanobody, mini-G or peptidomimetic binder; 2) at least one nanoluciferase comprising a LgBit and a SmBit; 3) a substrate; and 4) reagents and cells for protein expression. Additional kit components may optionally include, for example: 1) stabilizers and/or a buffer; 2) at least one container, vial or similar apparatus for holding and/or mixing the kit components; 3) a reagent transfer apparatus; and/or 4) instructions for using the components of the kit to perform a GPCR binding assay.

In some embodiments, the present invention provides a kit with a membrane-bound GPCR and a cytosolic nanobody-SmBit and SmBit-mini-G protein fusion protein. In some embodiments, in a kit with a membrane-bound GPCR, the cytosolic nanobody-SmBit and SmBit-mini-G protein fusion proteins are expressed separately for greater versatility to pair the GPCR with either a nanobody, a mini-G protein or a peptidomimetic. In some embodiments, a peptidomimetic is derived from the C-terminal helical domain of a G-proteins, or the chimeric form of multiple G-protein C-terminal helical domains, and/or comprises mutations or other unnatural amino acids.

In some embodiments, a kit of the present invention comprises positive and negative control reagents and ligands to identify possible sources of error and contamination. In some embodiments, a kit of the present invention comprises calibration reagents and ligands including, for example, a luminescence calibration reagent. In some embodiments, a kit of the present invention comprises a known GPCR assay with a same-class GPCR and same class ligand for comparison to a new ligand to be screened. In some embodiments, a kit provides a synthetic HiBit peptide to titrate with the GPCR-expressing cell pellet as a positive control. In the presence of the NANOLUC substrate, the HiBit reconstitutes with the LgBit in a cell pellet expressed and attached to a GPCR, and a luminescent signal confirms the correct expression of the protein.

In some embodiments, a kit comprises customizable options. In some embodiments, a kit of the present invention comprises frozen (e.g., prepared with glycerol for storing at −20 C degrees) mammalian cell pellets containing GPCRs expressed and attached to LgBit. To perform an activation screening in vitro, a client may order a kit with a GPCR(s) of choice. In addition to the cell pellet, the kit may include a corresponding binding partner, for example, a nanobody attached to SmBit or a peptidomimetic.

In some embodiments, a kit comprises:
1) flash frozen cell pellets expressing a GPCR of interest bound to LgBit;
2) separate frozen cell pellets expressing the GPCR of interest's corresponding binding partner bound to SmBit comprising, for example:
a) either a nanobody bound to SmBit; or
b) SmBit bound to mini-G protein; or
c) a solution of SmBit bound to a peptidomimetic;
3) buffer for resuspending a cell pellet;
4) a positive control containing cell pellet expressing beta 2 adrenergic receptor (B2AR)-LgBit; and
5) a cell pellet expressing NB80-SmBiT or SmBit bound to mini-Gs protein.

In some embodiments, a kit comprises a solution of SmBit bound to a peptidomimetic, and isoproterenol solution for activating 1 B2AR GPCR. The positive control may be provided to ensure that the cell pellet is well-stored and the protocol works.

In some embodiments, a kit comprises:
1) flash frozen cell pellets co-expressing a GPCR of interest bound to LgBit; and
2) SmBit bound to a GPCR's corresponding binding partner; comprising
   a) a nanobody; or
   b) mini G protein;
3) a buffer for resuspending the cell pellets; and
4) a positive control containing cell pellet co-expressing:
   a) B2AR-LgBit and NB80-SmBiT; or
   b) SmBit bound to mini-Gs protein; and
5) isoproterenol solution for activating B2AR.

In some embodiments, a kit of the present invention comprises:
1) a first cell lysate solution with 20-50% glycerol comprising the GPCR of interest bound to LgBit;
2) a second separate cell lysate solution with 20-50% glycerol comprising the GPCR of interest's corresponding binding partner bound to SmBit, comprising:
   a) either a nanobody bound to SmBit; or
   b) SmBit bound to mini-G protein; or
   c) a solution of SmBit bound to a peptidomimetic;
3) buffer for diluting the cell lysate;
4) a positive control containing cell lysate with 20-50% glycerol, comprising
   a) B2AR-LgBit and cell pellet expressing NB80-SmBiT; or
   b) SmBit bound to mini-Gs protein; or
   c) a solution of SmBit bound to a peptidomimetic; and
5) isoproterenol solution for activating B2AR.

In some embodiments, a kit of the present invention provides:
1) a cell lysate solution with 20-50% glycerol containing both a GPCR of interest bound to LgBit;
2) SmBit bound to a GPCR's corresponding binding partner; comprising:
   a) a nanobody; or
   b) a mini-G protein;
3) buffer for resuspending the cell pellet;
4) a positive control experiment containing cell lysate solution with 20-50% glycerol, containing:
   a) both B2AR-LgBit and NB80-SmBiT; or
   b) SmBit bound to mini-Gs protein; and
5) isoproterenol solution for activating B2AR.

In some embodiments, a positive control provides B2AR-LgBit and SmBit bound to Nb80, mini-Gs, and Gs-peptidomimetic with >2-fold isoproterenol (10 uM)-dependent luminescence increase, achieving statistical significance (p<0.05) using a student's T-test comparing the +isoproterenol condition to the no-drug vehicle group, indicating that the positive control experiment performs to specification.

### Compositions and Reaction Mixtures

The present disclosure provides compositions and reaction mixtures comprising components of the GPCR ligand binding assays described herein. Such compositions and reaction mixtures may comprise, for example, two or more of 1) at least one conformation specific nanobody, mini-G or peptidomimetic binder; 2) at least one nanoluciferase comprising a LgBit and SmBit; 3) a substrate; and 4) reagents and cells for protein expression. Additional composition and reaction mixture components may optionally include stabilizers and/or a buffer.

### EXPERIMENTAL EXAMPLES

The following examples are provided to demonstrate and further illustrate certain embodiments and aspects of the present disclosure and are not to be construed as limiting the scope thereof.

### Example 1—Split NANOLUC Assay in Cell Culture

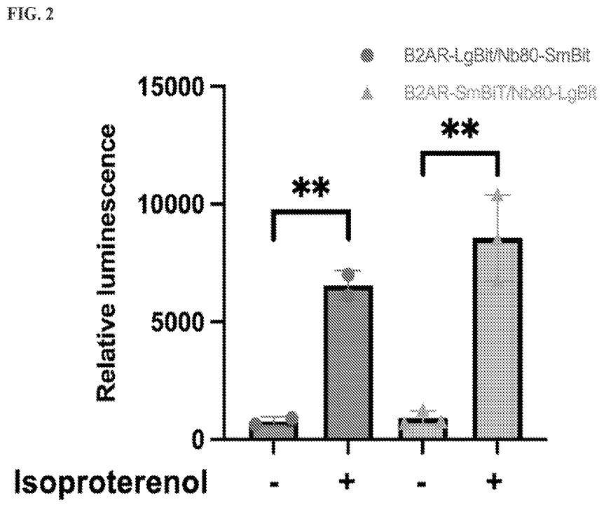

A NANOLUC assay in HEK293T cell culture was performed to screen diverse split NANOLUC fusion constructs to test whether orientation of the protein components affects the relative signal output (FIG. 1). The GPCR was attached to the LgBit fragment and the active-conformation-specific binding was attached to the SmBit and vice versa. (Soave, M.; Heukers, R.; Kellam, B.; Woolard, J.; Smit, M. J.; Briddon, S. J.; Hill, S. J. Monitoring Allosteric Interactions with CXCR4 Using NanoBiT Conjugated Nanobodies. Cell Chem. Biol. 2020, 27 (10), 1250-1261.e5.) Tests were performed in HEK293T cells using a model interaction protein pair of beta-2-adrenergic receptor (B2AR) and Nb80 which only binds to B2AR in the presence of an agonist such as isoproterenol (Iso). (Irannejad, R.; Tomshine, J. C.; Tomshine, J. R.; Chevalier, M.; Mahoney, J. P.; Steyaert, J.; Rasmussen, S. G. F.; Sunahara, R. K.; El-Samad, H.; Huang, B.; von Zastrow, M. Conformational Biosensors Reveal GPCR Signalling from Endosomes. Nature 2013, 495 (7442), 534-538, Ring, A. M.; Manglik, A.; Kruse, A. C.; Enos, M. D.; Weis, W. I.; Garcia, K. C.; Kobilka, B. K. Adrenaline-Activated Structure of B2-Adrenoceptor Stabilized by an Engineered Nanobody. Nature 2013, 502 (7472), 575-579.) Interaction of Nb80 and B2AR was tracked by the reconstitution of the split NANOLUC that generates a bioluminescent readout quantifiable by the plate-reader. Comparing the +Iso condition to the −Iso condition demonstrated a clear iso-dependent signal, indicating that binding and reconstitution of split NANOLUC only occurred in the drug-activated GPCR condition in cell culture (FIG. 2), and is independent of the geometry of the reconstitution in presence of the bioluminescent enzyme.

### Example 2—MOR-NB39 Split NANOLUC Assay in Cell Culture

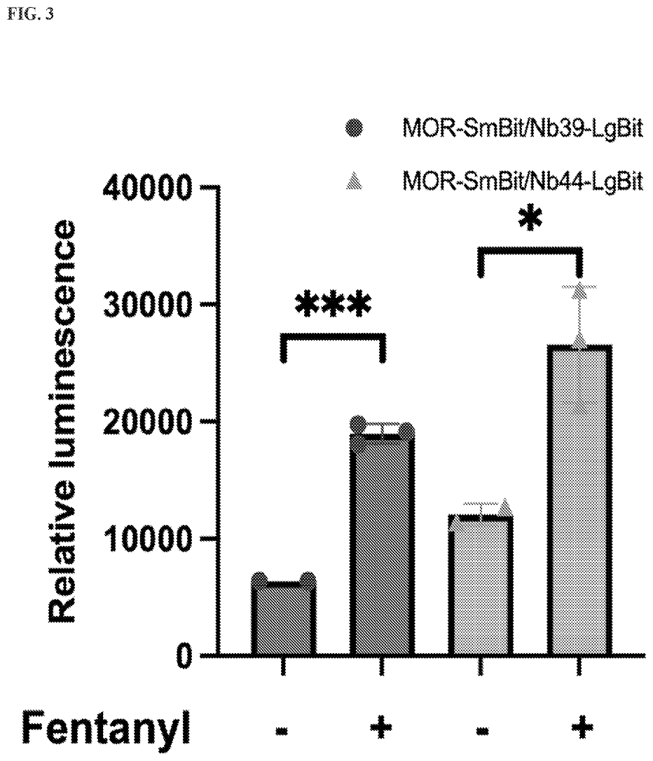

Reconstitution of split NANOLUC was used to track the binding of Nb39 to the Mu-opioid receptor (MOR). (Stoeber, M.; Jullié, D.; Lobingier, B. T.; Laeremans, T.; Steyaert, J.; Schiller, P. W.; Manglik, A.; von Zastrow, M. A Genetically Encoded Biosensor Reveals Location Bias of Opioid Drug Action. Neuron 2018, 98 (5), 963-976.e5, Vasudevan, L.; Stove, C. P. A Novel Nanobody-Based Bio-Assay Using Functional Complementation of a Split Nanoluciferase to Monitor Mu-Opioid Receptor Activation. Anal. Bioanal. Chem. 2020, 412 (29), 8015-8022.) The MOR was expressed with the SmBit attached to the C-terminus and Nb39 was expressed attached to LgBit on the C-terminus. Protein constructs were expressed in mammalian cell culture using the same method developed for the B2AR/Nb80 protein interacting partners above. Nb44 with binding affinity for the agonist-bound MOR was also tested. (Stoeber, M.; Jullié, D.; Lobingier, B. T.; Laeremans, T.; Steyaert, J.; Schiller, P. W.; Manglik, A.; von Zastrow, M. A Genetically Encoded Biosensor Reveals Location Bias of Opioid Drug Action. Neuron 2018, 98 (5), 963-976.e5.) As shown in FIG. 3, Nb39 and Nb44 both showed fentanyl opioid-dependence, demonstrating performance of the cell culture MOR-based split NANOLUC assay of the present invention.

### Example 3—B2AR-Based Split NANOLUC Assay In-Solution

Next, a NANOLUC assay in-solution assay was tested using the B2AR-based GPCR. (FIG. 1). To extract the GPCR from the membrane pellet, and to extract the cytosolic conformation-specific nanobody component from cell lysate, transfection of adherent HEK cells was used to express the protein components separately. Adhesion cell cultures transfected with protein components were harvested separately using a cell-scraper followed by suspension of the cells in Dulbecco's Phosphate Buffered Saline (DPBS) buffer. The pellet was washed with DPBS a second time, followed by cell lysis in a resuspension buffer containing HEPES, benzoase, and protease inhibitor. The membrane and cytosolic proteins are treated with respective buffers to preserve the stability of the protein components because they originate from distinct environments. To extract the membrane protein component, the insoluble membrane pellet was treated with 1% DDM (n-dodecyl β-D-maltoside) and 0.1% CHS (cholesteryl hydrogen succinate) membrane solubilization buffer, followed by sonication to extract the GPCRs from the membrane. For protein extraction of the soluble protein component containing the conformation specific nanobody, the cell pellet was treated with Mammalian Protein Extraction Reagent (M-PER) for cell lysis prior to sonication. After spinning down the lysed cells, the supernatant containing the soluble protein was preserved on ice until running the assay.

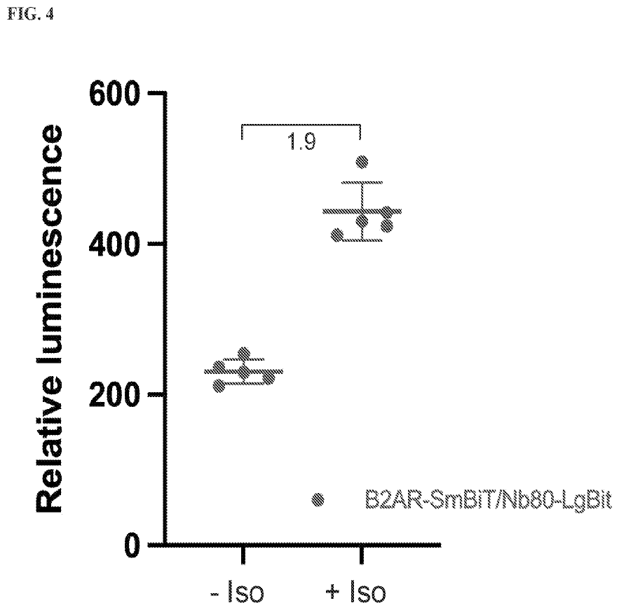

To test that the membrane protein component was folded correctly and able to be activated effectively by its agonist, the lysed solution was mixed with the cytosolic protein component solution. Initial testing used 10 uL of each solution added to a 96-well plate, followed by a brief incubation period with the agonist (˜10 μM per well). Substrate for the reconstituted NANOLUC is added in a buffer solution (1:19 substrate: buffer) as the final step before using the plate reader to detect and quantify the intensity of subsequent bioluminescence. If the well contained properly folded and functional membrane protein, the cytosolic protein (nanobody attached to LgBit) binds in the presence of the agonist, thereby allowing reconstitution of the split NANOLUC. As shown in FIG. 4 there was a 1.9-fold signal change between the (+Iso) and (−Iso) conditions indicating that the binding interaction between Nb80 and B2AR occurs in the presence of agonist. Accordingly, testing demonstrated use of the in-solution split NANOLUC assay for ascertaining the structural integrity of the GPCR, and use of the in-solution assay for ligand and drug screening.

### Example 4—MOR-Nanobody In-Solution Luminescence Assay Using HEK Cells for MOR Expression

MOR-SmBit fusion in HEK293T cells and extraction of the membrane-bound MOR protein using the DDM/CHS detergent-based solution are performed as developed for the B2AR-based in-solution assay. Both Nb39 and Nb44 are fused to the LgBit fragment, and the protein composite is expressed in HEK293T cells and extracted as the soluble cell extract. Western Blots are used to confirm the presence of MOR-SmBit in the membrane component extract and Nb39/Nb44-LgBit in the soluble protein extract. The split NANOLUC assay is tested using different ratios of the MOR-SmBit and Nb39/Nb44-LgBit protein mix to establish optimal conditions for generating a large opioid-dependent signal change.

To optimize the dynamic range (or signal-to-noise ratio SNR) of the opioid-dependent bioluminescent signal increase, the optimal ratio of the membrane protein component to the cytosolic protein component is tested. The ratios are adjusted to measure which component is the limiting factor. Mixing the cell lysates together thoroughly before aliquoting into the 96-well plate is used to ensure assay to assay consistency. The agonist-incubation period is tested to assess effects on the signal fold-change. The amount of time between addition of the NANOLUC substrate addition and the bioluminescence measurement using the plate reader is varied to establish an optimal time delay.

### Example 6—MOR and Nb Expression Using Suspension Cells for Large Scale Screening

### MOR Expression in Suspension Cells.

A limiting factor when using adherent mammalian cells to produce membrane proteins such as GPCRs is that the monolayer of cells grown in the flasks limits the quantity of proteins to be extracted, and also the scale of the in-solution experiments. The in-solution assay is optimized by selection of a stable cell line for expressing the GPCR membrane proteins in non-adherent K562 cells to scale expression and extraction of the GPCR proteins. K562 cells in suspension cells provide larger batch expression compared to the single layer of cells grown using, for example, HEK293T. Protein expression is measured using Western Blots, and the correlation between membrane protein concentration and signal fold-change is determined.

Nanobody Expression in E. coli.

To simultaneously screen multiple opioid agonists at a time, production of the Nanobody-LgBit components on a large scale and their storage for easy access is desired. To this end, the Nb39-LgBit is expressed in the periplasm of E. coli following established protocols. (Mannes, M.; Martin, C.; Triest, S.; Pia Dimmito, M.; Mollica, A.; Laeremans, T.; Menet, C. J.; Ballet, S. Development of Generic G Protein Peptidomimetics Able to Stabilize Active State Gₛ Protein-Coupled Receptors for Application in Drug Discovery. Angew. Chem. Int. Ed. 2021, 60 (18), 10247-10254.) The Nb39-LgBit protein is purified using a Ni-NTA column, followed by Fast Protein Liquid Chromatography using a size exclusion column. The purity of the nanobody component is measured by Sodium Dodecyl Sulfate-Polyacrylamide Gel Electrophoresis, and the concentration of the purified protein is determined by Bicinchoninic Acid (BCA) Protein Assay.

In-Solution Luminescent Assay with Different Concentrations of Protein Mix

Based on determined concentrations of MOR-SmBit, Nb39-LgBit, and Nb44-LgBit and the previously obtained optimal ratio of protein mix, assays with different concentrations of the split NANOLUC protein component mixture are evaluated to determine the assay's opioid-dependent fold change, and their opioid-sensitivity in correlation to the split NANOLUC protein concentrations. Opioid dose-response curves are generated from 0.01 nM to 1 mM opioids with GPCR-MOR concentrations at 0.1 nM, 1 nM, 10 nM, 100 nM, 1 μM, and 10 μM to identify the minimum protein concentrations needed to obtain robust opioid-dependent luminescent signal for drug screening.

This experimental example provides an in-solution luminescent assay to screen opioid-dependent MOR activation by its induced interaction with Nb39 and resulting reconstitution of split NANOLUC. Two protocols for large scale production of the MOR-SmBit protein from suspension K562 cells are provided, antecedent to the expression and purification of Nb39-LgBit protein from E. coli cells. The working concentration range of the extracted proteins is determined to establish the minimum required protein concentrations necessary to obtain a robust opioid-dependent signal. The resulting in-solution assay provides a consistent and robust method for screening candidate MOR agonists.

In some embodiments, insect cells are used for the production of the MOR-SmBit protein. (Rasmussen, S. G. F.; Choi, H.-J.; Fung, J. J.; Pardon, E.; Casarosa, P.; Chae, P. S.; DeVree, B. T.; Rosenbaum, D. M.; Thian, F. S.; Kobilka, T. S.; Schnapp, A.; Konetzki, I.; Sunahara, R. K.; Gellman, S. H.; Pautsch, A.; Steyaert, J.; Weis, W. I.; Kobilka, B. K. Structure of a Nanobody-Stabilized Active State of the B2 Adrenoceptor. Nature 2011, 469 (7329), 175-180.) In some embodiments, suspension mammalian cells are used to express Nb-LgBit.

### Example 7—In-Solution Split NANOLUC Assay Using Prefabricated Peptide Gₛ-Protein Peptidomimetics

In some embodiments, a G-protein peptidomimetic is provided as a conformation-specific binder. In some embodiments, a Gₛ-protein mimics and that binds with high affinity for GPCRs that couple to Gₛ-proteins is provided. (Mannes, M.; Martin, C.; Triest, S.; Pia Dimmito, M.; Mollica, A.; Laeremans, T.; Menet, C. J.; Ballet, S. Development of Generic G Protein Peptidomimetics Able to Stabilize Active State Gₛ Protein-Coupled Receptors for Application in Drug Discovery. Angew. Chem. Int. Ed 2021, 60 (18), 10247-10254.) The peptides are designed based on the conserved features of the α₅-helix of both the Gₛ-protein and the engineered mini-G protein bound to crystalized B2AR. (Mannes, ibid.) The α₅-helix interacts with a cavity between transmembrane domains 3 and 5 of B2AR after activation of the GPCR, comprising, for example, a stapled peptide (Ac-KKKFN_{c}[PraCRDAzk]IQRMHLRQYEChaL-OH) (SEQ ID NO.: 4). (Mannes, ibid.) In some embodiments, a peptide of the present invention comprises the unnatural amino acid cyclohexylalanine (Cha).

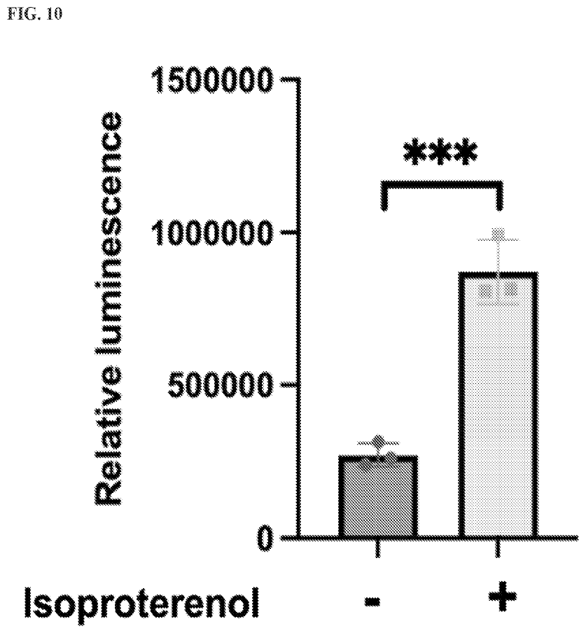

The Gₛ-protein mimic peptide is small and its generalizable for diverse Gₛ-GPCRs while still maintaining high affinity. In some embodiments, a protein component for an in-solution assay is generated using peptide synthesis of a Gₛ-mimic peptide (22 amino acids) fused with the SmBit peptide (11 amino acids) thereby eliminated the need for separately expressing a second protein component for the in-solution assays of the present invention. To streamline the synthesis of the hybrid peptide, we used the unstapled peptidomimetic that contains an unnatural amino acid (VTGYRLFEEILGSFNDCRDIIQRMHLRQYE-{Cha}-L) (SEQ ID NO.: 2). The SmBit portion of the hybrid peptide was tested for the capacity to reconstitute with extracted B2AR-LgBit. The membrane bound proteins were extracted using the membrane solubilization method described above (see “Testing split NANOLUC in-solution”. Cell lysate containing the fusion B2AR-LgBit protein was mixed with 10 μM of the hybrid SmBit-Gs-mimic peptide, and then assayed in a 96-well plate. The conditions were tested both with and without addition of B2AR agonist, isoproterenol (Iso). Furimazine substrate for NANOLUC was added last immediately before plate reading to quantify the bioluminescence. As shown in FIG. 10, a 3.2-fold signal change was observed between the +Iso and −Iso conditions, indicating that the Gₛ-mimic preferentially binds to Iso-activated B2AR, and that the SmBit portion of the hybrid peptide was able to successfully reconstitute with the LgBit in the cell lysate. These results demonstrate use of the Gₛ-mimic peptide approach to an in-solution assay for characterizing GPCR activation.

### Example 8—In-Solution Split NANOLUC Assay for GPCRs

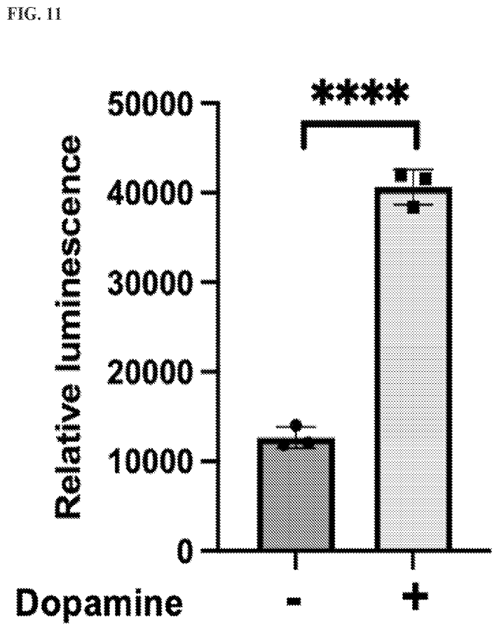

In some embodiments, Gₛ-peptide mimic-based in-solution luminescent assays are of use in determining the activation state of diverse Gₛ-coupled GPCRs. The versatility of the Gₛ-mimic peptide-based is tested assay using additional Gₛ-coupled GPCRs. For example, the Dopamine Receptor D₁ (DRD1) is fused to LgBit to evaluate dopamine-dependent bioluminescence (FIG. 11). The assay is optimized using different ratios of GPCR to peptide, and at different concentrations, to establish a preferred ratio of GPCR to peptide, and the minimum informative GPCR concentrations.

Increased versatility of the in-solution split NANOLUC assay of the present invention for diverse GPCRs is provided through modification of the existing Gₛ-mimic peptide to recognize other G-protein coupled GPCRs, including Gᵢ, G_{q}, and G_{12/13}-coupled GPCRs, in an agonist dependent manner. Analysis of the binding interaction between the α5-helix of these G-proteins and the GPCR interface from the available crystal structures indicates that the last 5 amino acids of the α5-helix are important to confer specificity for different G-proteins. For example in a method termed Dynamic Cyan Induction by Functional Integrated Receptors (DCyFIR) replacement of the terminal 5 amino acids of the G-protein α5-helix changes its specificity for different types of GPCRs. (Kapolka, N. J.; Taghon, G. J.; Rowe, J. B.; Morgan, W. M.; Enten, J. F.; Lambert, N. A.; Isom, D. G. DCyFIR: A High-Throughput CRISPR Platform for Multiplexed G Protein-Coupled Receptor Profiling and Ligand Discovery. Proc. Natl. Acad. Sci. 2020, 117 (23), 13117.)

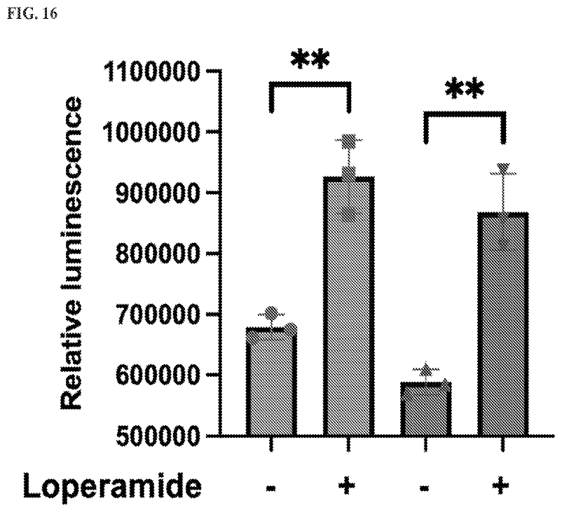

To test a Gᵢ-mimic peptide, a peptide was tested with incorporation of the unnatural amino acid, Cha, at the penultimate position of the peptide mimic. A leucine (Leu) residue at the penultimate position of G-proteins is conserved in G-proteins, indicating its importance in binding to GPCRs. (Mannes, ibid.) Changing Leu to Cha improves the binding affinity of Gₛ-protein mimic for B2AR. We therefore incorporated Cha to the Gi peptidomimetic with sequence: VTGYRLFEEILGSKKKFDCVTDCIIKNNLKDSGLF (SEQ ID NO.: 5). The cysteine mutations were initially introduced to install disulfide bond to stabilize the peptidomimetics. However, we found that the disulfide bond formation made the G protein peptidomimetics less selective for the active conformation of the GPCR. Therefore, when testing this Gi peptidomimetic, we added DTT to reduce the disulfide bond. FIG. 16 shows that this Gi peptidomimetic selectively binds to the agonist-bound MOR, enhancing the in-solution luminescence. To further test whether Cha replacement enhances the binding affinity for Gi protein mimics is tested for Gi peptidomimetics: peptide 1: VTGYRLFEEILGSKKKFDAVTDIIIKNNLKDCGLF (SEQ ID NO.: 6) and peptide 2: VTGYRLFEEILGSKKKFDAVTDIIIKNNLKDCG-{Cha}-F (SEQ ID NO.: 3) (Kapolka, ibid.), and compared in the in-solution luminescent assay together with the extracted MOR-LgBit fusion protein. In some embodiments, G-protein mimics for other G-proteins are synthesized to generate a panel of G-protein peptidomimetics for multiple GPCRs to support an in-solution split NANOLUC assay. G-protein mimics are tested using at least two GPCRs in each category to validate the generality of the peptidomimetic.

### Example 9—In-Solution Assays for GPCR Proteins

Current assays for evaluating the correct folding of extracted GPCRs mainly rely on the radioligand binding assay. (Rasmussen, S. G. F.; Choi, H.-J.; Fung, J. J.; Pardon, E.; Casarosa, P.; Chae, P. S.; DeVree, B. T.; Rosenbaum, D. M.; Thian, F. S.; Kobilka, T. S.; Schnapp, A.; Konetzki, I.; Sunahara, R. K.; Gellman, S. H.; Pautsch, A.; Steyaert, J.; Weis, W. I.; Kobilka, B. K. Structure of a Nanobody-Stabilized Active State of the B2 Adrenoceptor. Nature 2011, 469 (7329), 175-180.) However, the ligand binding assay is tedious and difficult to perform in a high-throughput manner. Additionally, the ligand binding assay does not detect whether the GPCR is able to confer the agonist-induced conformational change that leads to G-protein binding, and is not suitable for high-throughput analysis. To ensure the correct folding and functionality of the extracted GPCR, high-throughput luminescent assays are needed to characterize the reconstituted GPCRs. (Dixon, A. S.; Schwinn, M. K.; Hall, M. P.; Zimmerman, K.; Otto, P.; Lubben, T. H.; Butler, B. L.; Binkowski, B. F.; Machleidt, T.; Kirkland, T. A.; Wood, M. G.; Eggers, C. T.; Encell, L. P.; Wood, K. V. NANOLUC Complementation Reporter Optimized for Accurate Measurement of Protein Interactions in Cells. ACS Chem. Biol. 2016, 11 (2), 400-408.) As shown in FIG. 1, on agonist binding, the Gₛ-mimic peptide interacts with agonist bound-GPCR, and brings the split NANOLUC halves into proximity for split NANOLUC reconstitution, hence leading to luminescence. In-solution assays of the present invention support characterization of the correct-folding and functionality of the extracted GPCR in a high-throughput manner.

In some embodiments, the present invention provides an agonist-dependent fold change of at least 1.5-fold for in-solution assays to achieve a robust readout. In some embodiments, Z′ values are calculated by comparing the positive and negative binding results with multiple replicates to obtain a Z′ value >0.5, thereby indicating robustness of the assay for drug and ligand screening, and confirmation of the structural integrity of the extracted GPCR.

In some embodiments, mini-G proteins are used instead of and/or in addition to peptidomimetics. (Horing, C.; Seibel, U.; Tropmann, K.; Grätz, L.; Mönnich, D.; Pitzl, S.; Bernhardt, G.; Pockes, S.; Strasser, A. A Dynamic, Split-Luciferase-Based Mini-G Protein Sensor to Functionally Characterize Ligands at All Four Histamine Receptor Subtypes. Int. J. Mol. Sci. 2020, 21 (22), 8440, Carpenter, B.; Tate, C. G. Engineering a Minimal G Protein to Facilitate Crystallisation of G Protein-Coupled Receptors in Their Active Conformation. Protein Eng. Des. Sel. 2016, 29 (12), 583-594.) Mini-G proteins are smaller versions of their G-protein counterparts to bind to corresponding GPCRs. (Nehmé, R.; Carpenter, B.; Singhal, A.; Strege, A.; Edwards, P. C.; White, C. F.; Du, H.; Grisshammer, R.; Tate, C. G. Mini-G Proteins: Novel Tools for Studying GPCRs in Their Active Conformation. PLoS ONE 2017, 12 (4), e0175642.) By virtue of their size and stabilizing effects. Mini-G proteins are candidates for expression and co-crystallization. Similar to the nanobody-based assay, the LgBit-mini-G or SmBit-mini-G fusion protein may be expressed, purified, and stored separately.

### Example 10—In Vitro GPCR Activation Using Nanobodies

### Transfection, Growth and Harvest of GPCR Protein Pellets Using HEK293 Adhesion Cells

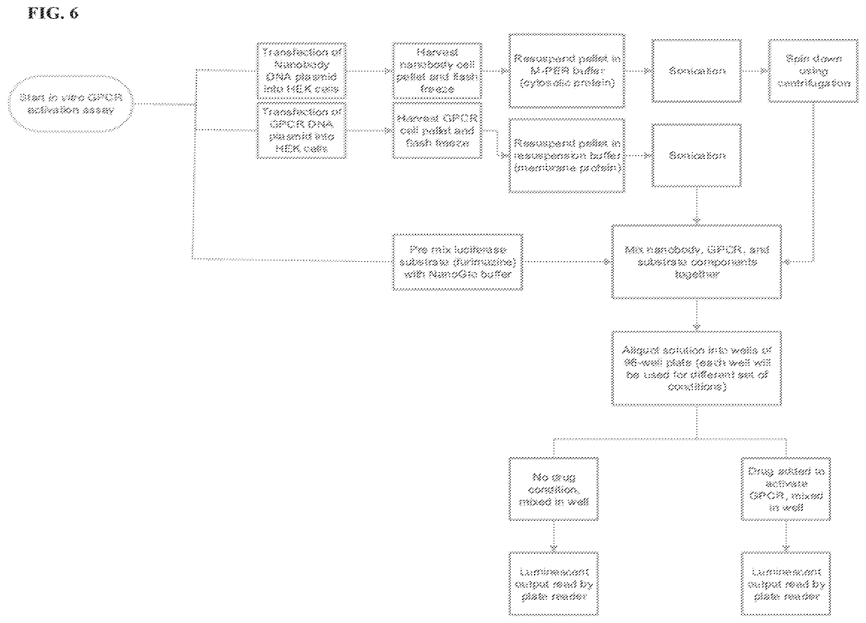

Bulk growth of HEK293 cell culture was performed in T-150 flask to 80-100% confluence (FIG. 6). A second T-150 flask was treated with human fibronectin (HFN) and incubated for 10 minutes. Split and plated confluent cells to 80-100% confluence were placed in the second flask. After 1-hour incubation, cells were transfected with FBS-free minimal essential medium (MEM), polyethylenimine (PEI), and 15 ug of plasmid DNA comprising a GPCR construct. The transfected HEK293 cells were incubated overnight for 18-24 hours. Cell pellets were harvested with a cell-scraper in Dulbecco's Phosphate Buffered Saline (DPBS) buffer. For a T-150 flask, the total volume for resuspending the cell pellets was 18 mL divided into 6 3 mL aliquots. The aliquots were centrifuged at 4,000 rpm for 4 minutes. The supernatant was discarded followed by resuspension of the cell pellets in 1 mL DPBS, and transfer to 1.5 mL Eppendorf tubes. The Eppendorf tubes were centrifuged at 8,000 rpm for 2 minutes. After the buffer was aspirated the pellets were flash-frozen in liquid nitrogen and stored in −80° C. freezer. In some embodiments, protein expression is provided by culture of other mammalian cells in culture and suspension, and use of stable cell lines. Protein expression is not limited to the method described herein.

### Assay Protein Preparation

### GPCR Membrane Protein Constructs

Cell pellets were thawed on ice and resuspended in 200 uL membrane resuspension buffer (resuspension buffer comprising 20 mM HEPES, pH 7.5, 2 mM MgCl2, protease inhibitor (50×) and benzoase (2 uL/5 mL of buffer); 4.5 mL resuspension buffer, and 45 uL protease inhibitor, benzonase 1.8 uL. The solution was then sonicated at an amplitude of 20 (3×1 sec pulse) and immediately placed on ice.

### Cytosolic Nanobody Protein Constructs

Cell pellets were thawed on ice and resuspended in 200 uL mammalian protein extraction reagent (M-PER) buffer. The solution was then sonicated at an amplitude of 20 (3×1 sec pulse). The sample was placed on ice before transferring for centrifugation at 10,000 rpm for 10 minutes, and placed on ice until use.

### GPCR Activation Assay

To prepare the Nano-Glo Live Cell Assay reagents (Promega, Madison, WI), a buffer and substrate was mixed at a 19:1 ratio in an amount sufficient to provide a target set of protein conditions. For example, with one GPCR and Nanobody protein combination to test, an amount sufficient for 7 reactions at 20 uL per reaction=133 uL buffer/7 uL furimazine was prepared to comprise 6 replicates for include 3 wells each for +/− test drug or ligand, and one well to compensate for possible lost volume.

The protein mixture was then added to the 140 uL of buffer+substrate. 70 uL of each protein component was added i.e., 70 uL of membrane protein cell lysate+70 uL of nanobody cytosolic protein in the supernatant. The total volume added was 140 uL sufficient for 10 uL of each protein component to be aliquoted per well i.e., 20 uL for both protein components together with thorough mixing throughout.

The total volume of the mixture was 280 uL partitioned into 7 wells to yield the final volume of 40 uL to aliquot into the 96-well plate. After 6 wells were filled with 40 uL each, the the activator drug/ligand was added to the “+drug” wells/conditions. For example, if the GPCR is β2AR, the activating drug was isoproterenol (Iso). The stock solution of the drug was 20 uM, and 20 uL of the stock solution was added to the “+drug” wells. For the “-drug” wells, 20 uL of the membrane resuspension buffer was added to maintain a standardized volume. The total concentration of Iso in the wells was 6.67 uM.

Optimization revealed that 8 minutes of incubation time is needed for the maximal luminescent signal output. After this interval, the 96-well plate was placed in the BioTek plate-reader (BioTek Instruments, Winooski, Vermont). An auto gain luminescence protocol measured the relative luminescence from each well before providing the final readings. Data output was processed and graphed using Prism 9.

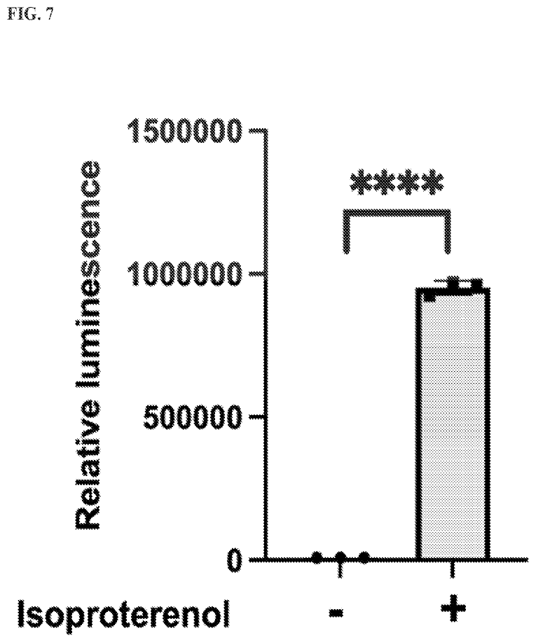

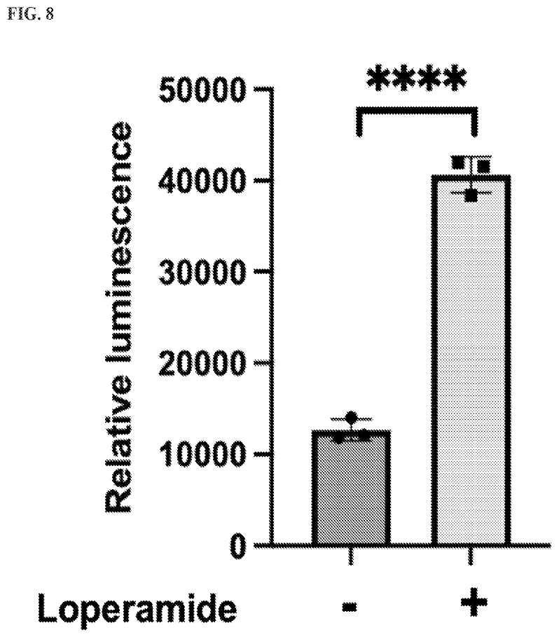

Graphs in the FIGS. 7 and 8 show that the signal output for 2 different GPCR/nanobody combinations i.e., activated B2AR binding to nanobody 80 (FIG. 7), and activated Mu-opioid receptor (MOR) binding to nanobody 39 (FIG. 8). The signal-to-noise (S/N) ratio was significant in both examples indicated by unpaired T-test analysis.

### Example 11—In Vitro GPCR Activation Using Peptidomimetics

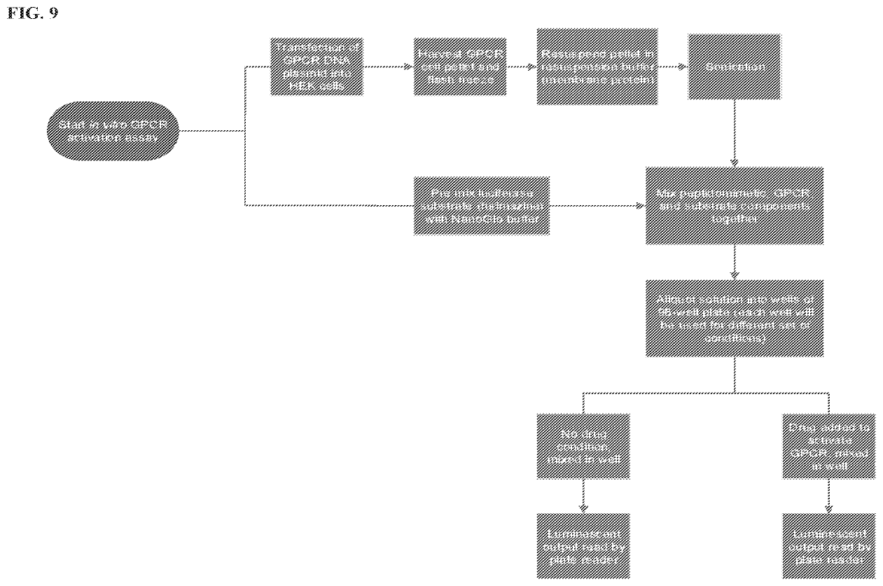

GPCR membrane protein steps of the assay were as described above i.e., the buffer and substrate of the Promega Nano-Glo Live Cell Assay reagents were mixed in a 19:1 ratio. (FIG. 9.) Sufficient Nano-Glo buffer and substrate were mixed for one target set of protein conditions. For one GPCR and peptidomimetic combination tested sufficient buffer and substrate were mixed for 7 reactions at 20 uL per reaction=133 uL buffer+7 uL furimazine.

Next, the protein mixture was added to the 140 uL of buffer+substrate. 70 uL of GPCR membrane protein cell lysate was mixed into the solution. The peptidomimetic, was stored as other small molecule or peptide drugs as stock solutions frozen in the −80° freezer. (LifeTein, Somerset, New Jersey) 17.5 uL of 100 uM stock solution was added to the assay mixture with an equal volume (17.5 uL) of membrane resuspension buffer to provide [35 uM] per well. The added peptidomimetic brought the total volume to 245 uL i.e., 7 samples at 35 uL per well.

After aliquoting 35 uL per well into the 96-well plate the activating ligand/drug was added. If the GPCR is β2AR, the activating drug was isoproterenol (Iso). 15 uL of the 20 uM was added to the “+drug” wells. For the “-drug” wells, 15 uL of membrane resuspension buffer was added to maintain a standardized volume. The total Iso concentration per well was 6 uM.

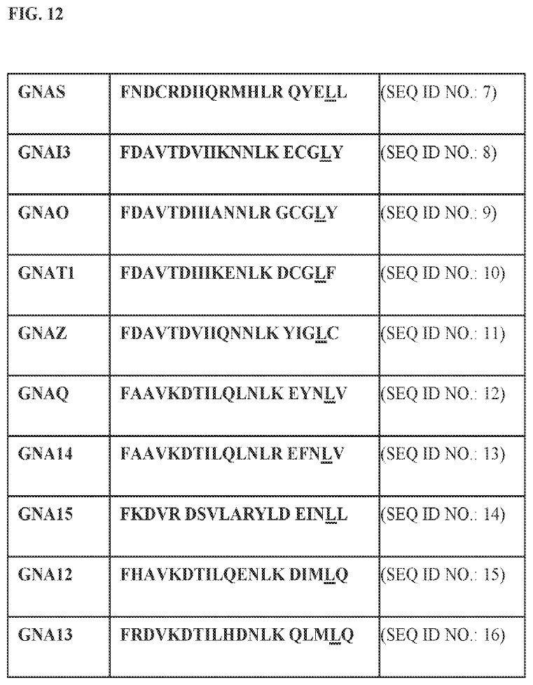

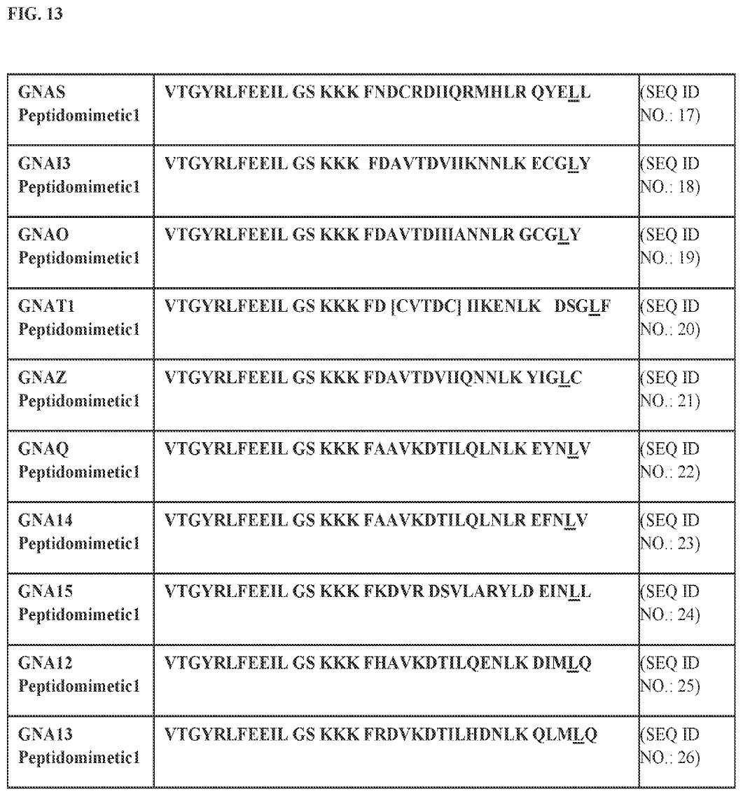

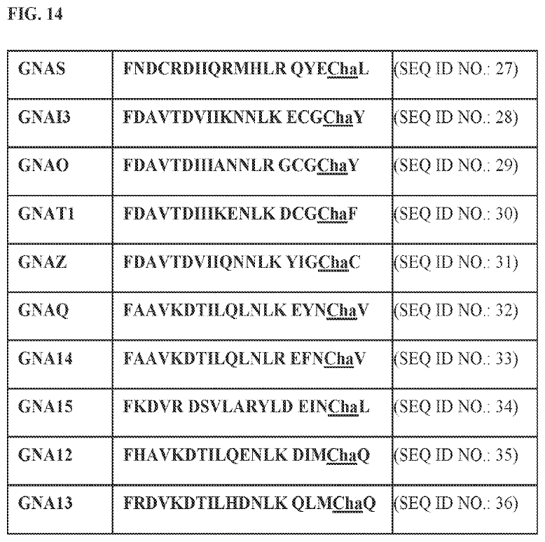

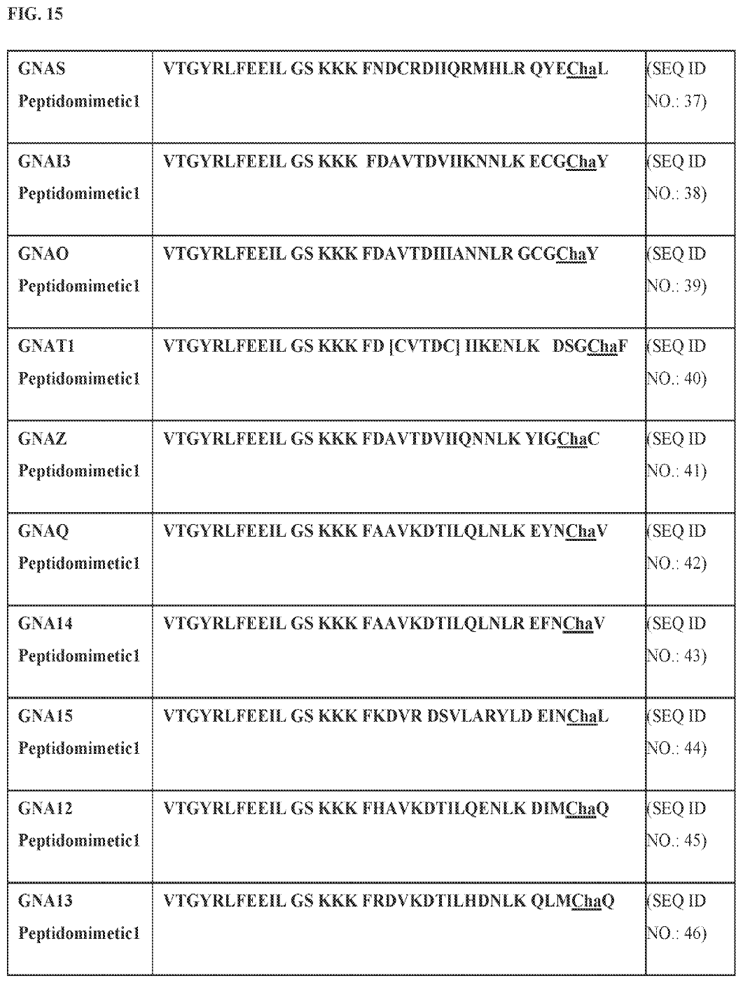

Optimization revealed that 8 minutes of incubation time is needed for maximal luminescent signal output. After this interval, the 96-well plate was placed inside the BioTek plate-reader. (BioTek Instruments, Winooski, Vermont) An auto gain luminescence protocol measured the relative luminescence from each well before providing the final readings. Data output was processed and graphed using Prism 9. FIG. 10 shows activation of the β-2AR with the Gs-peptidomimetic in both − and + isoproterenol conditions. FIG. 11 shows dopamine receptor D1 (DRD1) with the peptidomimetic in both − and + dopamine conditions. In both cases, the signal-to-noise ratio was significant, as demonstrated by the unpaired T-test. Representative amino acid sequences of peptidomimetics of the present invention are provided in FIGS. 12-15.

### Example 12—Co-Transfected MOR-Based Split NANOLUC In-Solution

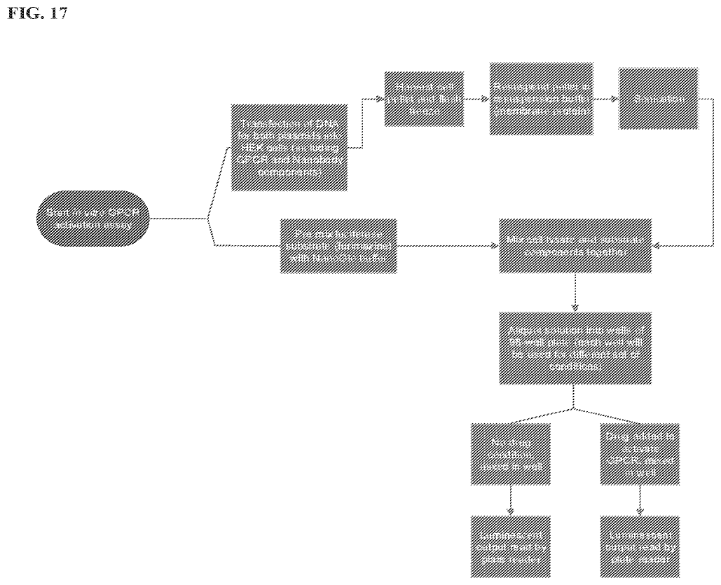

A co-transfected version of the NANOLUC assay in-solution assay was tested using the MOR-based GPCR. (FIG. 17). To simultaneously extract both the GPCR from the membrane pellet, and the cytosolic conformation-specific nanobody component (Nb39) from cell lysate, transfection of adherent HEK cells was used to express the protein components together in the same cells. Adhesion cell cultures transfected with both protein components were harvested using a cell-scraper followed by suspension of the cells in Dulbecco's Phosphate Buffered Saline (DPBS) buffer. The pellet was washed with DPBS a second time, followed by flash freezing the pellet in liquid nitrogen. After thawing the cell pellet, sonication was used for cell lysis in a resuspension buffer containing HEPES, benzoase, and protease inhibitor.

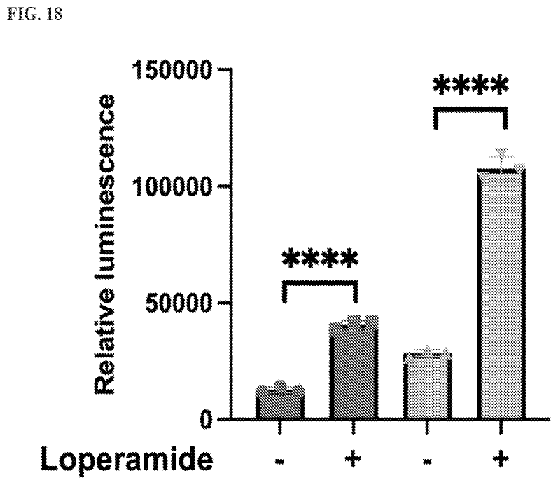

To test that the membrane protein component was folded correctly and able to be activated effectively by its agonist, 20 uL of the complete lysed solution was added to a 96-well plate, followed by a brief incubation period with the MOR agonist, Loperamide (˜10 μM per well). Substrate for the reconstituted NANOLUC is added in a buffer solution (1:19 substrate: buffer) as a final step before using the plate reader to detect and quantify the intensity of subsequent bioluminescence. If the well contains properly folded and functional membrane protein, the cytosolic protein (i.e., Nb39 linked to LgBit) binds in the presence of the agonist, thereby allowing reconstitution of the split NANOLUC. As shown in FIG. 18, a 3.2-fold signal change for the separate expression between the (+Lop) and (−Lop) conditions was observed indicating that the binding interaction between Nb39 and MOR occurs in the presence of agonist. The co-expressed version of the assay revealed a 3.8-fold signal change between the (+Lop) and (−Lop) conditions, that is greater than the separately expressed version. Accordingly, both the co-expressed and separately expressed components for the in-solution split NANOLUC assay find use for ascertaining structural integrity of the GPCR.

### Example 13—IGNiTR Assay

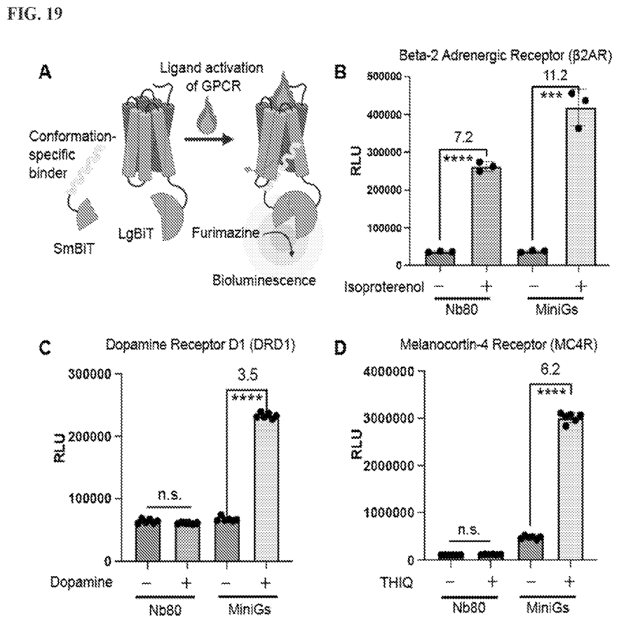

As shown in FIG. 19, IGNiTR comprises of two components: the GPCR fused to one half of the split NANOLUC, and a conformation specific binder fused to the other half of the enzyme. We used three different Gαₛ-coupled GPCRs as models to develop IGNiTR: β2-adrenergic Receptor (β2AR), Dopamine Receptor D1 (DRD1), and Melanocortin-4 Receptor (MC4R). (Israeli, H. et al. Structure reveals the activation mechanism of the MC4 receptor to initiate satiation signaling. Science 372, 808-814 (2021).) For the conformation-specific binding components, we used nanobodies and mini-Gₛ which have been used in live cell-based split NANOLUC assays. (Wan, Q. et al. Mini G protein probes for active G protein-coupled receptors (GPCRs) in live cells. Journal of Biological Chemistry 293, 7466-7473 (2018).) We tested Nanobody 80 (Nb80), which specifically binds activated β2AR. (Manglik, A., Kobilka, B. K. & Steyaert, J. Nanobodies to Study G Protein-Coupled Receptor Structure and Function. Annu. Rev. Pharmacol. Toxicol. 57, 19-37 (2017), Nehmé, R. et al. Mini-G proteins: Novel tools for studying GPCRs in their active conformation. PLoS ONE 12, e0175642 (2017), Rasmussen, S. G. F. et al. Crystal structure of the β2 adrenergic receptor-Gs protein complex. Nature 477, 549-555 (2011). Ring, A. M. et al. Adrenaline-activated structures β2-adrenoceptor stabilized by an engineered nanobody. Nature 502, 575-579 (2013).) For a generalizable application, we tested miniGₛ protein which has been shown to bind effectively to a range of the activated Gₛ-coupled GPCRs.

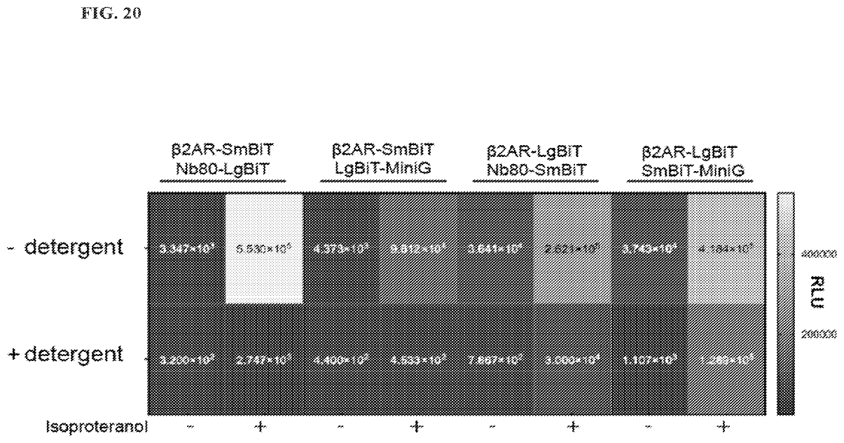

All IGNiTR constructs (prepared via sonication, FIG. 20) produced significant ligand-dependent luminescence increase, hence a value >1 for the ratio of the IGNiTR luminescence with drug to that without drug, abbreviated as drug dependent ratio (DDR). We expressed the GPCRs in mammalian cells to facilitate correct folding, membrane trafficking and post-translational modification of these complex proteins. (Wiseman, D. N.; Otchere, A.; Patel, J. H.; Uddin, R.; Pollock, N. L.; Routledge, S. J.; Rothnie, A. J.; Slack, C.; Poyner, D. R.; Bill, R. M.; et al. Expression and purification of recombinant G protein-coupled receptors: A review. Protein Expression and Purification 2020, 167, 105524.) Cell lysis is required for the G-protein mimic to access the intracellular loops of the GPCR and to enable storage of the cell pellet and ensure homogeneity of the lysate. Various methods have been developed for breaking up the cell membranes, as well as for solubilizing and stabilizing the GPCR protein, including the use of specific detergent mixes. (Urner, L. H.; Liko, I.; Yen, H.-Y.; Hoi, K.-K.; Bolla, J. R.; Gault, J.; Almeida, F. G.; Schweder, M.-P.; Shutin, D.; Ehrmann, S.; et al. Modular detergents tailor the purification and structural analysis of membrane proteins including G-protein coupled receptors. Nat Commun 2020, 11 (1), 564.) To maximally preserve GPCR protein folding and function, we tested two cell lysis conditions. We began by testing sonication of the cells in detergent-free solutions, since the native plasma membrane lipid environment provides crucial support for the structural integrity of membrane-bound GPCRs. (Jones, A. J. Y.; Gabriel, F.; Tandale, A.; Nietlispach, D. Structure and Dynamics of GPCRs in Lipid Membranes: Physical Principles and Experimental Approaches. Molecules 2020, 25 (20), Baccouch, R.; Rascol, E.; Stoklosa, K.; Alves, I. D. The role of the lipid environment in the activity of G protein coupled receptors. Biophysical Chemistry 2022, 285, 106794.) We also tested sonication in solutions containing a detergent mix frequently used to separate GPCR proteins from the membrane.

We tested two different fusion geometries with β2-adrenergic Receptor (β2AR) fused to either the large portion of the split NANOLUC, LgBiT, or the small portion, SmBit, and the G-protein mimic fused to the other half of the split NANOLUC. As shown in FIG. 20, significant ligand-dependent luminescence increase was observed for both Nb80 and miniGₛ in the two geometries in the lysis condition without detergent.

Data in FIG. 20 also indicates that different fusions affect IGNiTR performance. For example, when using Nb80 as the conformation-specific binder, much higher DDR was detected when GPCR was fused to SmBiT and Nb80 to LgBit. However, when using miniGₛ as the conformation specific binder, the opposite fusion geometry with GPCR fused to LgBiT and miniGₛ to SmBiT yielded a greater DDR.

FIG. 20 also shows that lysis with solutions containing detergent significantly diminish the luminescence in all conditions tested, suggesting the detergent disrupts the GPCR's functionality keeping with perturbations that affect the ability of the protein to be activated. (Manglik, A.; Kobilka, B. K.; Steyaert, J. Nanobodies to Study G Protein-Coupled Receptor Structure and Function. Annu. Rev. Pharmacol. Toxicol. 2017, 57 (1), 19-37. DOI: 10.1146/annurev-pharmtox-010716-104710.) This experiment highlights the importance of keeping the GPCRs in their native lipid environment. Therefore, for optimal IGNiTR assay performance, the cell pellet will be lysed by sonication without detergent.

IGNiTR composed of β2AR fused to LgBiT and SmBiT fused to Nb80 or miniGₛ each yielded significant DDRs (FIG. 19B), indicating Nb80 and miniGₛ can both selectively bind to the active conformation of β2AR in cell lysate. As expected, Nb80 did not show significant DDR with either DRD1 or MC4R. While when using miniGₛ, both DRD1 and MC4R produced significant DDRs (FIGS. 19C and 19D). The results indicate that IGNiTR detects a GPCR's agonist-dependent conformational change in cell lysate. In turn, these results indicate that miniGₛ is applicable for Gₛ-coupled GPCRs.

### Example 14—Using G-Protein Peptidomimetics in IGNiTR

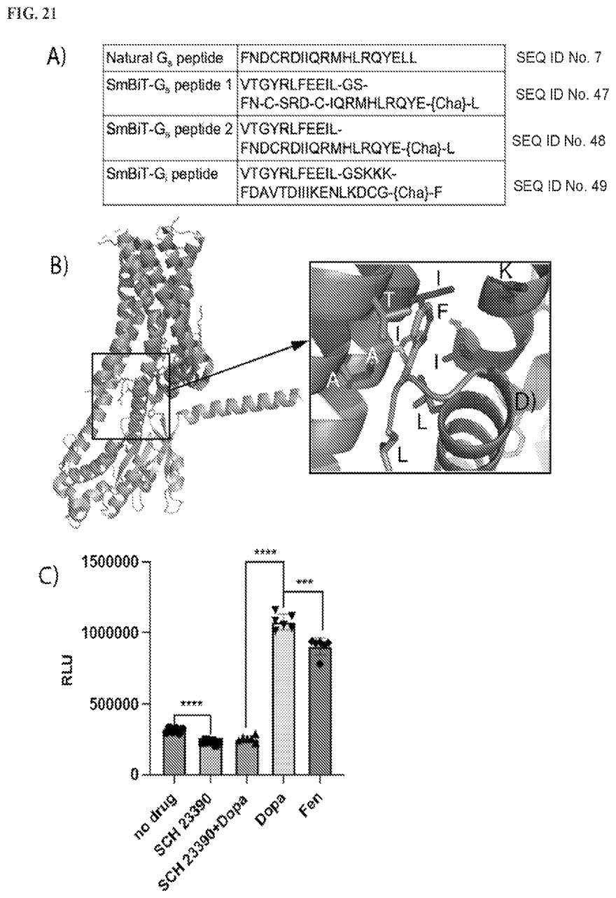

To increase the versatility of IGNiTR, we used a G-protein peptidomimetic as the conformation-specific binder in IGNiTR (FIG. 21). Peptidomimetics enable the use of unnatural amino acids to increase binding affinity for the GPCR target. (Mannes, M. et al. Development of Generic G Protein Peptidomimetics Able to Stabilize Active State G <sub>s</sub> Protein-Coupled Receptors for Application in Drug Discovery. Angew. Chem. Int. Ed. 60, 10247-10254 (2021). Boyhus, L.-E. et al. G <sub>s</sub> protein peptidomimetics as allosteric modulators of the β <sub>2</sub>-adrenergic receptor. RSC Adv. 8, 2219-2228 (2018).) Additionally, peptide synthesis facilitates the standardization of peptidomimetic concentration across large batches of well plates Our design was inspired by a reported Gₛ peptidomimetic (Mannes, M. et al. Development of Generic G Protein Peptidomimetics Able to Stabilize Active State G <sub>s</sub> Protein-Coupled Receptors for Application in Drug Discovery. Angew. Chem. Int. Ed 60, 10247-10254 (2021).) (FIG. 21), which was based on the α-5-helix of Gαₛ in the crystal structure of the Gₛ protein complex bound with β2AR. (Carpenter, B., Nehmé, R., Warne, T., Leslie, A. G. W. & Tate, C. G. Structure of the adenosine A2A receptor bound to an engineered G protein. Nature 536, 104-107 (2016).) Another version of the reported G, peptidomimetic incorporated two unnatural amino acids in the helical backbone away from the GPCR contact sites that form covalent bonds to generate stapled peptides to stabilize the helical structure. We tested both the non-stapled and this stapled G, peptidomimetic (FIG. 21). For the stapled peptidomimetics, we tested using cysteine residues instead to facilitate a di-sulfide bridge formation in the α-helix. We designed two fusion peptides composed of SmBiT fused to the two versions of Gₛ peptidomimetics (FIG. 21) and tested these fusion peptides in the IGNiTR assay using β2AR. While the disulfide-bond containing peptide 1 did not demonstrate agonist-dependence, Gₛ peptidomimetic peptide 2 showed agonist-dependent DDR. The high background signal of Gₛ peptide 1 can be attributed to the cysteine-based disulfide bond failing to connect the helical structure of the α-5-helix. The G, peptidomimetic (FNDCRDIIQRMHLRQYE-[Cha]-L) conserves the Gαₛ protein α-5-helix amino acid sequence while adding a cyclohexylalanine (Cha) residue to increase hydrophobic interactions with the large hydrophobic pocket of the activated β2AR. (Gumpper, R. H. & Roth, B. L. G-Protein Peptidomimetics Stabilize GPCR Active State Conformations. Trends in Pharmacological Sciences 42, 429-430 (2021).)

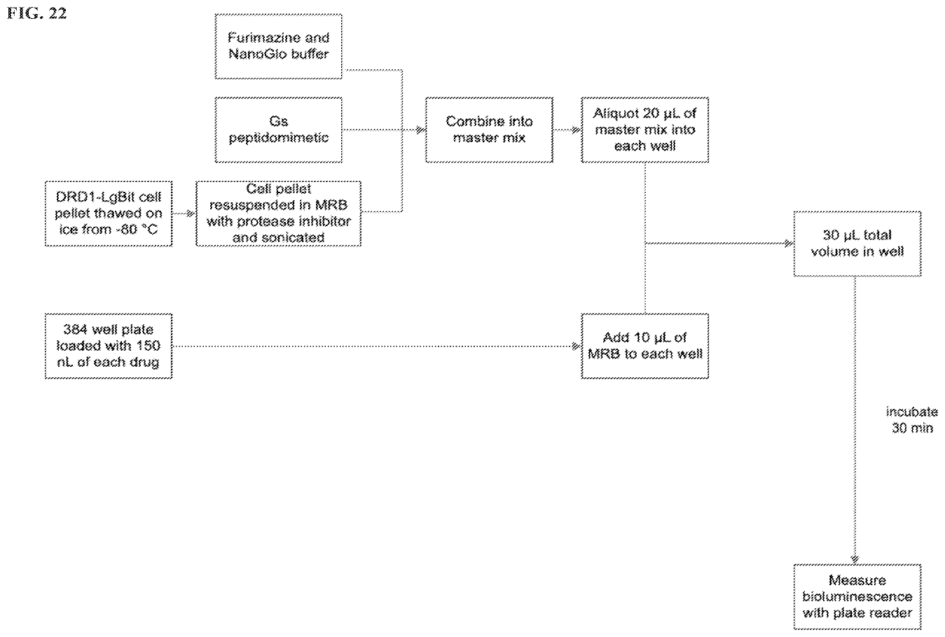

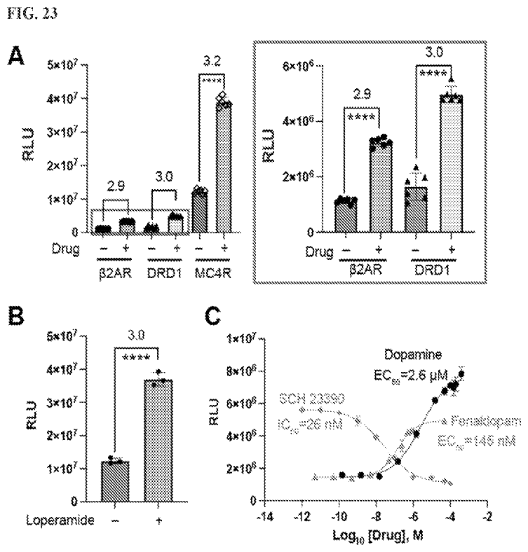

We fused the SmBiT (11 amino acids) to the G, peptidomimetic to create a SmBiT-G, peptidomimetic fusion peptide. To test the peptidomimetic version of IGNiTR, β2AR-LgBiT protein in sonicated cell lysate was mixed with the G, fusion peptide and NANOLUC substrate. Then, agonist or vehicle was added to evaluate the DDR (FIG. 22). β2AR IGNiTR with the Gₛ fusion peptide produced a significant DDR. We further tested the Gₛ fusion peptide with the other two Gₛ-coupled GPCRs, MC4R and DRD1, each producing significant DDRs (FIG. 23A). These results validated the Gₛ peptidomimetic's selectivity for the active conformation of the Gₛ-coupled GPCRs.

We then designed a Gᵢ peptidomimetic using a parallel strategy because the α-5-helix of the Gαᵢ-protein also interacts with a highly hydrophobic binding pocket based on the Gαᵢ-protein structure. (Koehl, A. et al. Structure of the μ-opioid receptor-Gi protein complex. Nature 558, 547-552 (2018).) The Gᵢ fusion peptide is composed of the SmBiT fused to the Gᵢ peptidomimetic. We tested IGNiTR with the Gᵢ fusion peptide and a Gᵢ-coupled GPCR, the μ-opioid receptor (μ-OR). A significant DDR was observed for μ-OR IGNiTR (FIG. 23B) thereby validating the Gᵢ peptide's selective binding to the agonist-activated μ-OR and establishes the use of Gᵢ fusion peptide in IGNiTR for Gᵢ-coupled GPCRs.

### Example 15—IGNiTR Assay to Characterize GPCR Ligand Efficacy and Potency

To establish IGNiTR's ability to characterize the diverse conformational states of a GPCR induced by various ligands, we applied the technique to DRD1 IGNiTR with full agonists, partial agonists and antagonists. The full agonist dopamine produced higher DDR than the partial agonist fenoldopam at saturated concentrations, with both producing a DDR>1. The result validates that both full and partial agonists induce the active conformational state (Teng, X. et al. Ligand recognition and biased agonism of the D1 dopamine receptor. Nat Commun 13, 3186 (2022), Teng, X. et al. Structural insights into G protein activation by D1 dopamine receptor. Sci. Adv. 8, eabo4158 (2022).), and that IGNiTR differentiates ligand efficacies. DRD1 antagonist, SCH 23390, does not increase luminescence compared to the no drug condition. These results further validate the Gₛ peptidomimetic's selective binding to the active conformation of DRD1. As well, DRD1 titration with dopamine and fenoldopam produced EC50 values of 2.6 μM and 145 nM, respectively (FIG. 23C) that correspond with the reported EC50 values. DRD1 titration with antagonist SCH 23390 in the presence of 10 μM agonist dopamine yielded an IC50 of 26 nM, which also agrees with the reported value. (Kumar, M., Hsiao, K., Vidugiriene, J. & Goueli, S. A. A Bioluminescent-Based, HTS-Compatible Assay to Monitor G-Protein-Coupled Receptor Modulation of Cellular Cyclic AMP. ASSAY and Drug Development Technologies 5, 237-246 (2007).) These characterizations demonstrate that the GPCR in IGNiTR maintains function comparable to live cell assays and that IGNiTR differentiates between diverse ligand efficacies.

### Example 16—IGNiTR Application: High Throughput Screening (HTS) with Robust Z′ Values

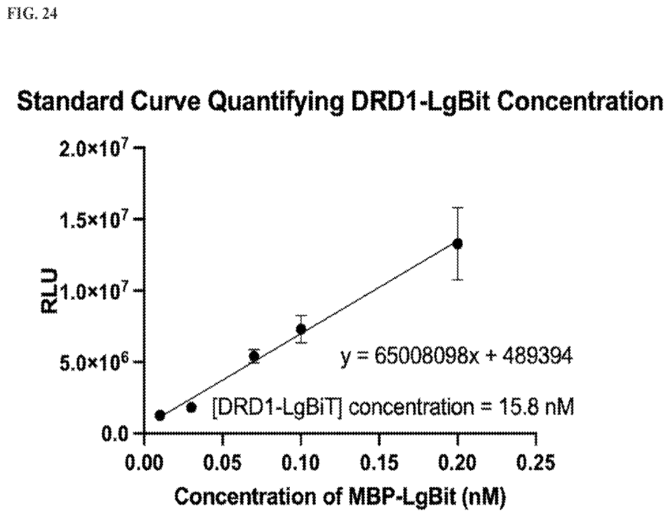

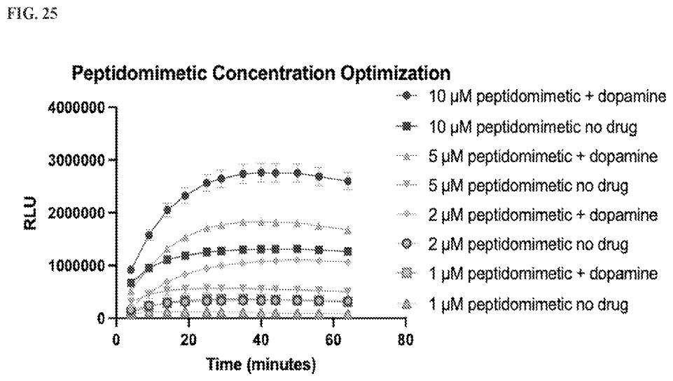

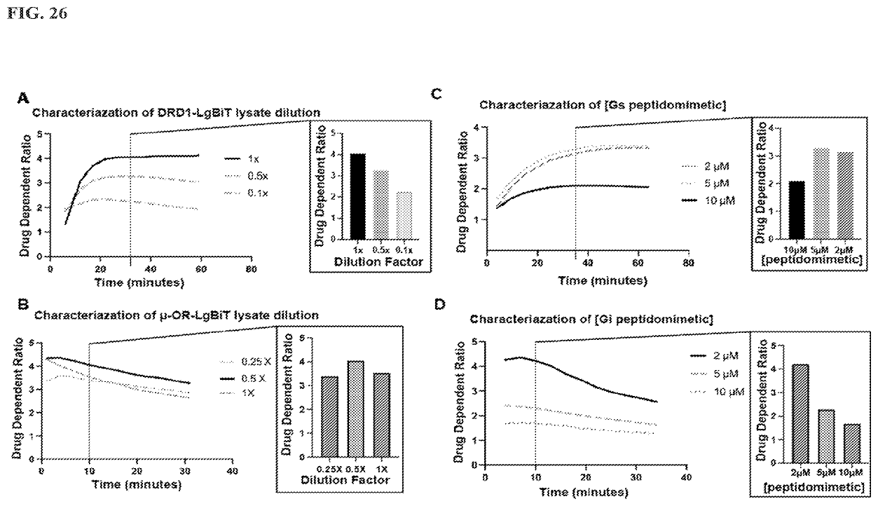

In some embodiments, IGNiTR provides an alternative for HTS of GPCR ligands, especially because IGNiTR components may be mixed in a batch, increasing consistency across large-scale screens. We optimized DRD1 IGNiTR assay conditions by varying the DRD1 and G, fusion peptide concentrations (FIGS. 24-26). An advantage of an in vitro assay is the ability to modulate the concentration of each of the components in the assay and fine tune the conditions. We therefore characterized the performance of the IGNiTR assay under following conditions: GPCR-LgBiT concentration, ligand incubation time, and Gₛ fusion peptide concentration. To characterize IGNiTR with different GPCR-LgBiT concentrations, we first estimated the relative concentration of GPCR-LgBiT by creating a standard curve of LgBiT with its high-affinity peptide partner, HiBiT (Schwinn, M. K.; Machleidt, T.; Zimmerman, K.; Eggers, C. T.; Dixon, A. S.; Hurst, R.; Hall, M. P.; Encell, L. P.; Binkowski, B. F.; Wood, K. V. CRISPR-Mediated Tagging of Endogenous Proteins with a Luminescent Peptide. ACS Chem. Biol. 2018, 13 (2), 467-474, C. W.; Kilpatrick, L. E.; Pfleger, K. D. G.; Hill, S. J. A nanoluciferase biosensor to investigate endogenous chemokine secretion and receptor binding. iScience 2021, 24 (1), 102011.) (FIG. 24). We then varied the dilution factor for the GPCR-LgBiT cell lysate while the peptidomimetic concentration was held constant at 10 μM. Among the dilutions, the 1× and 0.5× dilutions yielded the best DDR of >3 when measured at 30 minutes for DRD1 (FIGS. 25 and 26). Because the signal stabilizes around 25-30 minutes after ligand incubation, we measured the luminescence at 30 minutes for all DRD1 characterizations. For the μ-OR-LgBiT, we observed that the DDR ratio peaks around 10 minutes, with all three dilutions, 0.25×, 0.5× and 1×, producing comparable DDR of ˜4 (FIG. 26).

The IGNiTR assay was then characterized with different concentrations of the fusion Gₛ and Gᵢ fusion peptides. To minimize the cell pellet needed for characterization, we used the 0.5× of the GPCR-LgBiT lysate dilutions. We varied the concentration of fusion peptides added. For both Gₛ and Gᵢ fusion peptides, the highest concentration (10 μM) resulted in a lower DDR compared to the lower concentrations due to higher background luminescence (FIG. 26). Drug-dependent DDR ratios of ˜3 were observed with 5 μM and 2 μM G_{αs} fusion peptidomimetic and the highest DDR of ˜4 was observed with 2 μM Gᵢ peptidomimetic. We therefore used the 2 μM fusion peptide in the subsequent DRD1 and μ-OR IGNiTR assays to maximize DDR and to reduce the volume of fusion peptides needed for high throughput screening (HTS).

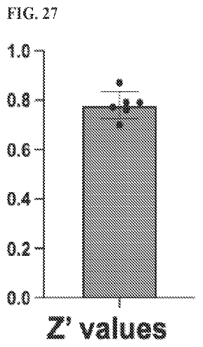

The optimized DRD1-IGNiTR assay was then used to scale up to screen for potential agonists using 1,916 compounds from an FDA-approved & Passed Phase I Drug Library from SelleckChem library. The Z′ value was consistent across the plates with an average of 0.79 (FIG. 27) which is within the range of optimal Z′ value for HTS (1>Z′>0.5). (Zhang, J.-H., Chung, T. D. Y. & Oldenburg, K. R. A Simple Statistical Parameter for Use in Evaluation and Validation of High Throughput Screening Assays. SLAS Discovery 4, 67-73 (1999).) Even though no hit molecules were identified from this library of compounds, this proof-of-principle screening demonstrated the feasibility and robustness of IGNiTR in GPCR ligand screening.

### Example 17—IGNiTR Rapid Detection of Opioids Outside of a Laboratory

In some embodiments, IGNiTR is packaged as an accessible kit for detecting GPCR agonists, such as opioids, outside of a biosafety level 2 laboratory space. The ongoing opioid crisis is being fueled by the emergence of additional synthetic opioids. (Cuitavi, J., Hipólito, L. & Canals, M. The Life Cycle of the Mu-Opioid Receptor. Trends in Biochemical Sciences 46, 315-328 (2021). Mattson, C. L. et al. Trends and Geographic Patterns in Drug and Synthetic Opioid Overdose Deaths—United States, 2013-2019. MMWR Morb Mortal Wkly Rep 70, 202-207 (2021).) Accordingly, there is a pressing need for accessible methods to detect opioid derivatives which often are highly potent and thus have the potential to cause lethal overdoses. To address this need, we tested using μ-OR-based IGNiTR to detect the synthetic opioid, fentanyl.

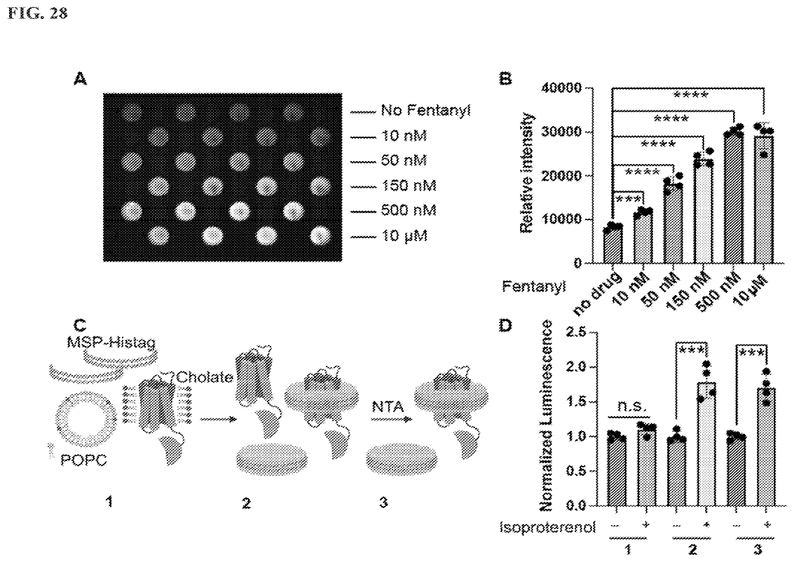

First, we optimized the μ-OR and Gᵢ fusion peptide concentrations for μ-OR IGNiTR (FIG. 26 B and D). To increase accessibility of IGNiTR for detection, we measured IGNiTR luminescence with a less sophisticated gel-imaging camera, rather than a plate reader. As shown in FIG. 28 A and B, higher concentrations of fentanyl result in increased luminescence intensity, with a plateau around 500 nM. μ-OR-based IGNiTR reliably detected 10 nM fentanyl which produced a significantly different intensity compared to the no drug control. These results demonstrate that IGNiTR successfully detects diverse levels of opioids. Notably, IGNiTR reports on the general presence of opioids, which complements existing assays for detecting specific synthetic opioid molecules. (Glasscott, M. W. et al. Electrochemical sensors for the detection of fentanyl and its analogs: Foundations and recent advances. TrAC Trends in Analytical Chemistry 132, 116037 (2020), Razlansari, M. et al. Nanobiosensors for detection of opioids: A review of latest advancements. European Journal of Pharmaceutics and Biopharmaceutics 179, 79-94 (2022).) Because the IGNiTR reagents are readily frozen and stored, in some embodiments the components of IGNiTR are packaged into a kit for detecting μ-OR agonists in a diversity of settings. Further, IGNiTR is readily adaptable to detect other GPCR agonists enabling biosensor development for a wide range of molecules.

### Example 18—Characterizing GPCR Functionality During IGNiTR Nanodisc-Based GPCR Extraction and Reconstitution

Nanodiscs are widely applied for GPCR reconstitution by embedding the GPCR in a lipid bilayer, forming stable GPCR-lipid complexes. (Rouck, J. E., Krapf, J. E., Roy, J., Huff, H. C. & Das, A. Recent advances in nanodisc technology for membrane protein studies (2012-2017). FEBS Letters 591, 2057-2088 (2017).) It remains critical but challenging to track the GPCR structural integrity and function throughout the Nanodisc assembly process. (Serebryany, E., Zhu, G. A. & Yan, E. C. Y. Artificial membrane-like environments for in vitro studies of purified G-protein coupled receptors. Biochimica et Biophysica Acta (BBA)—Biomembranes 1818, 225-233 (2012).) Therefore, we tested IGNiTR's ability to characterize β2AR functionality during the three crucial steps of POPC-based Nanodisc formation (Mitra, N. et al. Calcium-Dependent Ligand Binding and G-protein Signaling of Family B GPCR Parathyroid Hormone 1 Receptor Purified in Nanodiscs. ACS Chem. Biol. 8, 617-625 (2013).) as indicated in FIG. 28C. Higher DDR indicates greater content of functional β2AR that can undergo agonist-dependent conformational changes, binding to the Gₛ peptidomimetic and reconstituting the split NANOLUC. As shown in FIG. 28D, β2AR reconstituted in Nanodisc with detergent cholate removed (sample 2), and its subsequent Ni-NTA purified sample (sample 3), produced a significant agonist-dependent DDRs, where the β2AR mixed with Nanodisc components as well as cholate (sample 1) did not yield a significant DDR. This result validates the importance of removing cholate for the correct folding and functionality of β2AR during its incorporation into the Nanodisc. These data support use of IGNiTR to monitor GPCR functionality throughout the protein extraction and reconstitution process in order to optimize protocols.

### Materials and Methods

### DNA Constructs and Cloning

Standard cloning procedures, including NEB restriction enzyme digest, Q5 polymerase PCR amplification, T4 ligation and Gibson assembly, were used. Oligonucleotide primers were purchased from Sigma Aldrich. The plasmid DNA encoding MC4R was a gift from the Roger Cone Lab. The plasmid DNA encoding MiniGs and Nanobody 80 was purchased from Twist Biosciences.

Plasmid constructs were transformed into Escherichia coli cells via heat shock. XL1-Blue competent cells were used for all constructs, except for MBP-LgBit, which is described in “Expression and Purification of MBP-LgBit” below. Sequences were confirmed by Sanger Sequencing (Eurofins, GeneWiz)

### Cell Culture and Transfections

HEK 293T/17 cell lines (ATCC, cat #: CRL-11268) used in these experiments were cultured at 37° C. and 5% CO₂. Cells were grown in complete growth media (1:1 MEM (Eagle's Minimal Essential Medium): DMEM (Dulbecco's Modified Eagle Medium, Gibco): with 50 mM HEPES (Gibco), 10% Fetal Bovine Serum (Sigma), and 1% Penicillin-Streptomycin (Gibco).

Cells at 80-90% confluence were plated into a flask that had been pre-incubated with human fibronectin for 10 minutes at 37° C. An hour after seeding, these cells were transfected using FBS-free MEM and polyethylenimine (PEI, 1 mg/ml, Polysciences) with a ratio of 1:10 between μL of PEI and pg of plasmid DNA. The cells were then incubated at 37° C. for 20-24 hours.

The cell pellet was harvested by aspirating the media and resuspending the cells with a cell scraper in Dulbecco's Phosphate Buffered Saline (DPBS) buffer. 18 mL of DPBS was used to resuspend the cells within a T-75 flask, with this ratio kept constant for other flask sizes. 1.5 mL of resuspended cells were placed into an Eppendorf tube, which was centrifuged at 6,010 g for 3 minutes. The supernatant was aspirated, and the pellet was resuspended in DPBS, then centrifuged again under the same conditions. The supernatant was aspirated again, and the cell pellet was flash frozen in liquid N₂ and stored at −80° C. until ready for use.

### Preparation of Cell Pellet

The cell pellets were prepared immediately before the assay, by first placing the cell pellet onto ice. For cell pellets containing GPCR constructs, the pellet was treated with 210 μL of Membrane Resuspension Buffer (MRB). MRB is comprised of incomplete membrane resuspension buffer (resuspension buffer comprising 20 mM HEPES, pH 7.5 and 2 mM MgCl₂) and benzoase (EMD Millipore, 70746) in a 4.5 mL to 1.8 μL ratio. For cell pellets containing cytosolic proteins (Nanobody 80 or MiniGs), the pellet was treated with Mammalian Protein Extraction Reagent buffer. After this, 2 μL of 100× protease inhibitor (Sigma Aldrich, P1860 #) was added to a final concentration of 1× and the pellet was resuspended by pipetting up and down. The solution was then sonicated using Model 50 Sonic Dismembrator (Fischer Brand) at a 20% amplitude (3×1 sec pulse) and returned to ice.

### IGNiTR Assay

A master mix containing Nano-Glo Buffer and substrate (Promega, N2012), GPCR construct, and G protein mimic was prepared. For one well, the ratio was 9.75 μL of Nano-Glo Buffer, 0.25 μL of Nano-Glo substrate, 5 μL of GPCR pellet and 5 μL of the conformation specific binder. The conformation specific binder may be either peptidomimetic (LifeTein), miniGs pellet or Nanobody 80 pellet. The GPCR construct cell pellet was prepared separately and added to the master mix immediately prior to adding the master mix to the well. The concentrations were varied for the optimization assays (FIG. 4), but the volumes remained constant. Concentrations for each experiment are indicated in the figure legends. The 384 well cell culture plates were preloaded with 10 μL of drug per well, followed by loading of the 20 μL of the master mix per well. Times reported on figure captions were recorded from the addition of the master mix to the first well of the plate. The luminescence values for all the conditions were measured using an EnVision 2104 Multilabel Reader (Perkin Elmer).

### High Throughput Screening

To scale up the IGNiTR assay for high throughput screening, we used the Echo 655 (Labcyte) to load 150 nL of drug to each well of the 384 well plate. Then, the Multidrop Combi Reagent Dispenser (Thermo Scientific) was used to add 10 μL of MRB to each well. The same machine was used to add 20 μL of master mix, prepared as described in “IGNiTR Assay” using 2 μM fusion peptidomimetic and the 0.5× dilution of DRD1-LgBiT found to be optimal from the characterization in FIG. 4. After a 30-minute incubation, the luminescence values were measured using an EnVision 2104 Multilabel Reader (Perkin Elmer).

$$Corrected Relative Luminescence = \frac{\begin{matrix}luminescence of sample - \\ mean luminescence of negative control\end{matrix}}{\begin{matrix}mean luminescence of positive control - \\ mean luminescence of negative control\end{matrix}} \times 100 \%$$

We validated the drug hits from an initial screen of six 384 well plates by testing four replicates of each compounds using the same method. From this validation, a dose response curve was constructed for promising candidates by loading 150 nL at a range of concentrations, using a mosquito X1 (SPT Labtech) to obtain final concentrations in well from 661 nM to 25.1 μM. 10 μL of MRB was loaded to the compounds, followed by 20 μL of master mix. After a 30 minute incubation, the luminescence values were measured using an EnVision 2104 Multilabel Reader (Perkin Elmer).

### Expression and Purification of MBP-LgBiT

The DNA encoding MBP-LgBit was transformed into BL21 cells. A colony of these cells was inoculated in 5 mL Luria-Bertani broth with ampicillin at 37° C. overnight. The culture was then transferred to a 500 mL flask of Luria-Bertani broth with ampicillin and placed in a 37° C. shaker until OD-600 reached 0.4 to 0.8. Protein expression was induced by addition of 1000×0.1 g/mL IPTG, to a final concentration of 1×. The culture was then shaken overnight at room temperature.

The cells were centrifuged at 4,248 g for 5 minutes at 4° C. The cell pellet was lysed by resuspension in cold Bacterial Protein Extraction Reagent (B-PER, Fisher) buffer with 1 mM dithiothreitol (DTT) and 1× protease inhibitor (BioBasic, BS386). 15 mL of B-PER was used for every 500 mL of bacteria culture. 3-4 μL of benzoase was added to the cells, followed by a 5 min incubation on ice to ensure full cell lysis. The cells were then centrifuged at 16,994 g for 10 minutes at 4° C.

50 mL of clear lysate was added to 2 mL of Ni-NTA resin slurry and incubated at 4° C. for 10 minutes. This mixture was then purified via an Ni-NTA column. The purity of MBP-LgBiT was then established using gel electrophoresis and a Coomassie stain analysis.

### Determining the Concentration of MBP-LgBiT

The molar extinction coefficient of MBP-LgBiT was calculated using Expasy PratParam to be 89,270 M⁻¹ cm⁻¹. This value was then used in conjunction with Bier's Law to establish the concentration of the protein by the absorbance at A₂₈₀. The absorbance was re-calculated for each time MBP-LgBiT was used.

### Standard Curve

The standard curve was created using the same 384 well plates. First, a master mix containing a ratio of 5 μL of 30 μM HiBiT, 0.125 μL furimazine and 4.875 μL NanoGlo Buffer was prepared. 10 μL of this master mix was added to each well. 5 μL of MBP-LgBit or GPCR-LgBit was added, in the dilution ratio indicated in the figure legends. Times reported on figure captions were recorded from the addition of the MBP-LgBit or GPCR-LgBit to the first well of the plate.

### Statistical Analysis

All statistical analysis was performed using GraphPad Prism 9 software. This software was also used to construct plots. The sample size is indicated in figure legends (where n is the number of independent replicates). The mean and standard error of the mean were calculated for each condition. Two-sided Student's t-tests were used to evaluate the significance between data points. Z′ values were calculated using the following equation:

$${Z}^{′} = 1 - \frac{3 SD of positve control + 3 SD of negative control}{\vert mean of positive control - mean of negative control \vert}$$

where SD is the standard deviation.

### Fentanyl Detection Using IGNiTR

The frozen pellet expressing μ-OR-LgBiT was thawed, resuspended, and mixed with the Gᵢ fusion peptide and NANOLUC substrate. The reaction mix was aliquoted to separate wells of an opaque white 96-well plate. A range of concentrations of fentanyl were added to the wells and the plate was imaged using an Azure Biosystems c600 for chemiluminescence. The resulting images were analyzed using imageJ.

### Nanodisc Assembly and Purification

The pellet (β2AR-LgBiT) was resuspended in 400 uL membrane resuspension buffer (MRB) and sonicated. The lysate was quantified using the Pierce BCA protein assay kit (Thermo Scientific). The protein concentration was calculated using an average molecular weight of 40 kDa for membrane proteins. Nanodiscs were made as described previously. POPC (Avanti Polar Lipids) was dried under nitrogen and stored in a desiccator overnight. POPC was solubilized to 50 mM with 100 mM sodium cholate. Nanodiscs were assembled by adding MSP1E3D1 (Millipore Sigma) and lysate to the solubilized lipids up to a final volume of 350 μL in standard disc buffer (20 mM Tris, 100 mM NaCl, 0.5 mM EDTA, 0.01% NaN3) supplemented with sodium cholate to a final concentration of 20 mM. The final lysate concentration in the mixture was 10 uM, the MSP:lysate was 4:1, and the lipid:MSP was 90:1. The component mixture was incubated on an end over end mixer at 4° C. for 45 minutes. 150 mg of Amberlite XAD-2 beads (Millipore Sigma) were added, and the component mixture was incubated at 4° C. overnight before the beads were removed. The resulting Nanodiscs were then purified with Ni-NTA spin columns (NEB). The purified Nanodiscs were then exchanged into standard disc buffer with Bio-Spin P-6 Gel Columns (Bio-Rad) to remove imidazole.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

All publications, published patent documents, and patent applications cited herein are hereby incorporated by reference to the same extent as though each individual publication, published patent document, or patent application was specifically and individually indicated as being incorporated by reference.

We claim:

1. A method of measuring ligand activation of a G-protein coupled receptor (GPCR) in solution, comprising:
a) expressing a GPCR fusion protein comprising said GPCR and a first luciferase subunit in a cell;
b) extracting said GPCR fusion protein from said cell;
c) generating a reaction mixture in solution comprising said GPCR fusion protein with a conformation specific nanobody, wherein said conformation specific nanobody is bound to a second luciferase subunit that is complementary to said first subunit fused to said GPCR, wherein binding of said first and second luciferase subunits generates an intact luciferase, a bioluminescent substrate, and a buffer;
d) adding said ligand to said reaction mixture in said solution; and
e) measuring luminescence of said reaction mixture in said solution comprising said ligand wherein luminescence intensity indicates ligand activation of said GPCR.

2. The method of claim 1, wherein said GPCR comprises a beta 2-adrenergic receptor (B2AR) GPCR, a morphine receptor (MOR) GPCR, or a dopamine receptor D1 (DRD1) GPCR.

3. The method of claim 1, wherein said cell is a prokaryotic cell or a mammalian cell.

4. The method of claim 1, wherein said conformation specific nanobody comprises Nb80, Nb39 or Nb40.

5. The method of claim 1, wherein said first or second luciferase subunit is 17.5 kDa.

6. The method of claim 5, wherein the other of said first or second luciferase subunit comprises 11 amino acids.

7. A method of measuring ligand activation of a G-protein coupled receptor (GPCR) in solution, comprising:
a) expressing a GPCR fusion protein comprising said GPCR and a first luciferase subunit in a cell;
b) extracting said GPCR fusion protein from said cell;
c) generating a reaction mixture in solution comprising said GPCR fusion protein with a conformation specific peptidomimetic, wherein said conformation specific peptidomimetic is bound to a second luciferase subunit that is complementary to said first subunit fused to said GPCR, wherein binding of said first and second subunits generates an intact luciferase, a bioluminescent substrate, and a buffer;
d) adding said ligand to said reaction mixture in said solution; and
e) measuring luminescence of said reaction mixture in said solution comprising said ligand wherein luminescence intensity indicates ligand activation of said GPCR.

8. The method of claim 1, wherein said GPCR comprises a beta 2-adrenergic receptor (B2AR) GPCR, a morphine receptor (MOR) GPCR, or a dopamine receptor D1 (DRD1) GPCR.

9. The method of claim 1, wherein said cell is a prokaryotic cell or a mammalian cell.

10. The method of claim 7, wherein the conformation specific peptidomimetic is selected from the group consisting of SEQ ID NO.: 1 to SEQ ID NO.: 46.

11. The method of claim 7, wherein said first or second luciferase subunit is 17.5 kDa.

12. The method of claim 11, wherein the other of said first or second luciferase subunit comprises 11 amino acids.

13. A kit, comprising:
a) a GPCR fusion protein comprising said GPCR and a first luciferase subunit;
b) a conformation specific nanobody, wherein said conformation specific nanobody is bound to said first luciferase subunit or a second luciferase subunit complementary to said first luciferase subunit fused to said GPCR:
c) a bioluminescent substrate; and
d) a buffer.

14. The kit of claim 13, further comprising one or more test ligands and/or one or more control ligands.

15. A kit, comprising:
a) a GPCR fusion protein comprising said GPCR and a first luciferase subunit;
b) a conformation specific peptidomimetic, wherein said conformation specific peptidomimetic is bound to said first luciferase subunit or a second luciferase subunit complementary to said first luciferase subunit fused to said GPCR:
c) a bioluminescent substrate; and
d) a buffer

16. The kit of claim 15, further comprising one or more test ligands and/or one or more control ligands.

17. A composition, comprising:
a) GPCR fusion protein comprising said GPCR and a first luciferase subunit;
b) a conformation specific nanobody, wherein said conformation specific nanobody is bound to said first luciferase subunit or a second luciferase subunit complementary to said first luciferase subunit fused to said GPCR:
c) a bioluminescent substrate; and
d) a buffer.

18. The composition of claim 17, further comprising one or more test ligands and/or one or more control ligands.

19. A composition, comprising:
a) GPCR fusion protein comprising said GPCR and a first luciferase subunit;
b) a conformation specific peptidomimetic, wherein said conformation specific peptidomimetic is bound to said first luciferase subunit or a second luciferase subunit subunit complementary to said first luciferase subunit fused to said GPCR:
c) a bioluminescent substrate; and
d) a buffer.

20. The composition of claim 19, further comprising one or more test ligands and/or one or more control ligands.


SEQUENCE LISTING (49 sequences)

SEQ ID NO: 1 - protein, 35 aa, synthetic construct
VTGYRLFEEILGSKKKFNCSRDCIQRMHLRQYEXL

SEQ ID NO: 2 - protein, 32 aa, synthetic construct
VTGYRLFEEILGSFNDCRDIIQRMHLRQYEXL

SEQ ID NO: 3 - protein, 35 aa, synthetic construct
VTGYRLFEEILGSKKKFDAVTDIIIKNNLKDCGXF

SEQ ID NO: 4 - protein, 22 aa, synthetic construct
KKKFNXCRDXIQRMHLRQYEXL

SEQ ID NO: 5 - protein, 35 aa, synthetic construct
VTGYRLFEEILGSKKKFDCVTDCIIKNNLKDSGLF

SEQ ID NO: 6 - protein, 35 aa, synthetic construct
VTGYRLFEEILGSKKKFDAVTDIIIKNNLKDCGLF

SEQ ID NO: 7 - protein, 19 aa, synthetic construct
FNDCRDIIQRMHLRQYELL

SEQ ID NO: 8 - protein, 19 aa, synthetic construct
FDAVTDVIIKNNLKECGLY

SEQ ID NO: 9 - protein, 19 aa, synthetic construct
FDAVTDIIIANNLRGCGLY

SEQ ID NO: 10 - protein, 19 aa, synthetic construct
FDAVTDIIIKENLKDCGLF

SEQ ID NO: 11 - protein, 19 aa, synthetic construct
FDAVTDVIIQNNLKYIGLC

SEQ ID NO: 12 - protein, 19 aa, synthetic construct
FAAVKDTILQLNLKEYNLV

SEQ ID NO: 13 - protein, 19 aa, synthetic construct
FAAVKDTILQLNLREFNLV

SEQ ID NO: 14 - protein, 19 aa, synthetic construct
FKDVRDSVLARYLDEINLL

SEQ ID NO: 15 - protein, 19 aa, synthetic construct
FHAVKDTILQENLKDIMLQ

SEQ ID NO: 16 - protein, 19 aa, synthetic construct
FRDVKDTILHDNLKQLMLQ

SEQ ID NO: 17 - protein, 35 aa, synthetic construct
VTGYRLFEEILGSKKKFNDCRDIIQRMHLRQYELL

SEQ ID NO: 18 - protein, 35 aa, synthetic construct
VTGYRLFEEILGSKKKFDAVTDVIIKNNLKECGLY

SEQ ID NO: 19 - protein, 35 aa, synthetic construct
VTGYRLFEEILGSKKKFDAVTDIIIANNLRGCGLY

SEQ ID NO: 20 - protein, 35 aa, synthetic construct
VTGYRLFEEILGSKKKFDCVTDCIIKENLKDSGLF

SEQ ID NO: 21 - protein, 35 aa, synthetic construct
VTGYRLFEEILGSKKKFDAVTDVIIQNNLKYIGLC

SEQ ID NO: 22 - protein, 35 aa, synthetic construct
VTGYRLFEEILGSKKKFAAVKDTILQLNLKEYNLV

SEQ ID NO: 23 - protein, 35 aa, synthetic construct
VTGYRLFEEILGSKKKFAAVKDTILQLNLREFNLV

SEQ ID NO: 24 - protein, 35 aa, synthetic construct
VTGYRLFEEILGSKKKFKDVRDSVLARYLDEINLL

SEQ ID NO: 25 - protein, 35 aa, synthetic construct
VTGYRLFEEILGSKKKFHAVKDTILQENLKDIMLQ

SEQ ID NO: 26 - protein, 35 aa, synthetic construct
VTGYRLFEEILGSKKKFRDVKDTILHDNLKQLMLQ

SEQ ID NO: 27 - protein, 19 aa, synthetic construct
FNDCRDIIQRMHLRQYEXL

SEQ ID NO: 28 - protein, 19 aa, synthetic construct
FDAVTDVIIKNNLKECGXY

SEQ ID NO: 29 - protein, 19 aa, synthetic construct
FDAVTDIIIANNLRGCGXY

SEQ ID NO: 30 - protein, 19 aa, synthetic construct
FDAVTDIIIKENLKDCGXF

SEQ ID NO: 31 - protein, 19 aa, synthetic construct
FDAVTDVIIQNNLKYIGXC

SEQ ID NO: 32 - protein, 19 aa, synthetic construct
FAAVKDTILQLNLKEYNXV

SEQ ID NO: 33 - protein, 19 aa, synthetic construct
FAAVKDTILQLNLREFNXV

SEQ ID NO: 34 - protein, 19 aa, synthetic construct
FKDVRDSVLARYLDEINXL

SEQ ID NO: 35 - protein, 19 aa, synthetic construct
FHAVKDTILQENLKDIMXQ

SEQ ID NO: 36 - protein, 19 aa, synthetic construct
FRDVKDTILHDNLKQLMXQ

SEQ ID NO: 37 - protein, 35 aa, synthetic construct
VTGYRLFEEILGSKKKFNDCRDIIQRMHLRQYEXL

SEQ ID NO: 38 - protein, 35 aa, synthetic construct
VTGYRLFEEILGSKKKFDAVTDVIIKNNLKECGXY

SEQ ID NO: 39 - protein, 35 aa, synthetic construct
VTGYRLFEEILGSKKKFDAVTDIIIANNLRGCGXY

SEQ ID NO: 40 - protein, 35 aa, synthetic construct
VTGYRLFEEILGSKKKFDCVTDCIIKENLKDSGXF

SEQ ID NO: 41 - protein, 35 aa, synthetic construct
VTGYRLFEEILGSKKKFDAVTDVIIQNNLKYIGXC

SEQ ID NO: 42 - protein, 35 aa, synthetic construct
VTGYRLFEEILGSKKKFAAVKDTILQLNLKEYNXV

SEQ ID NO: 43 - protein, 35 aa, synthetic construct
VTGYRLFEEILGSKKKFAAVKDTILQLNLREFNXV

SEQ ID NO: 44 - protein, 35 aa, synthetic construct
VTGYRLFEEILGSKKKFKDVRDSVLARYLDEINXL

SEQ ID NO: 45 - protein, 35 aa, synthetic construct
VTGYRLFEEILGSKKKFHAVKDTILQENLKDIMXQ

SEQ ID NO: 46 - protein, 35 aa, synthetic construct
VTGYRLFEEILGSKKKFRDVKDTILHDNLKQLMXQ

SEQ ID NO: 47 - protein, 32 aa, synthetic construct
VTGYRLFEEILGSFNCSRDCIQRMHLRQYEXL

SEQ ID NO: 48 - protein, 30 aa, synthetic construct
VTGYRLFEEILFNDCRDIIQRMHLRQYEXL

SEQ ID NO: 49 - protein, 35 aa, synthetic construct
VTGYRLFEEILGSKKKFDAVTDIIIKENLKDCGXF